SEQUENCE LISTING (74 sequences)

SEQ ID NO: 1 - protein, 1 aa, Artificial sequence
  A carboxy terminal portion of a Vbeta-segment, belonging to one of 23 novel Vbeta groups
C

SEQ ID NO: 2 - protein, 2 aa, Artificial sequence
  A carboxy terminal portion of a Vbeta-segment, belonging to one of 23 novel Vbeta groups
CA

SEQ ID NO: 3 - protein, 2 aa, Artificial sequence
  A carboxy terminal portion of a Vbeta-segment, belonging to one of 23 novel Vbeta groups
CS

SEQ ID NO: 4 - protein, 3 aa, Artificial sequence
  A carboxy terminal portion of a Vbeta-segment, belonging to one of 23 novel Vbeta groups
CAS

SEQ ID NO: 5 - protein, 3 aa, Artificial sequence
  A carboxy terminal portion of a Vbeta-segment, belonging to one of 23 novel Vbeta groups
CAW

SEQ ID NO: 6 - protein, 3 aa, Artificial sequence
  A carboxy terminal portion of a Vbeta-segment, belonging to one of 23 novel Vbeta groups
CSA

SEQ ID NO: 7 - protein, 4 aa, Artificial sequence
  A carboxy terminal portion of a Vbeta-segment, belonging to one of 23 novel Vbeta groups
CASS

SEQ ID NO: 8 - protein, 4 aa, Artificial sequence
  A carboxy terminal portion of a Vbeta-segment, belonging to one of 23 novel Vbeta groups
CATS

SEQ ID NO: 9 - protein, 4 aa, Artificial sequence
  A carboxy terminal portion of a Vbeta-segment, belonging to one of 23 novel Vbeta groups
CAWS

SEQ ID NO: 10 - protein, 4 aa, Artificial sequence
  A carboxy terminal portion of a Vbeta-segment, belonging to one of 23 novel Vbeta groups
CSVE

SEQ ID NO: 11 - protein, 4 aa, Artificial sequence
  A carboxy terminal portion of a Vbeta-segment, belonging to one of 23 novel Vbeta groups
CSAR

SEQ ID NO: 12 - protein, 5 aa, Artificial sequence
  A carboxy terminal portion of a Vbeta-segment, belonging to one of 23 novel Vbeta groups
CASSL

SEQ ID NO: 13 - protein, 5 aa, Artificial sequence
  A carboxy terminal portion of a Vbeta-segment, belonging to one of 23 novel Vbeta groups
CASSQ

SEQ ID NO: 14 - protein, 5 aa, Artificial sequence
  A carboxy terminal portion of a Vbeta-segment, belonging to one of 23 novel Vbeta groups
CASSY

SEQ ID NO: 15 - protein, 5 aa, Artificial sequence
  A carboxy terminal portion of a Vbeta-segment, belonging to one of 23 novel Vbeta groups
CASSE

SEQ ID NO: 16 - protein, 5 aa, Artificial sequence
  A carboxy terminal portion of a Vbeta-segment, belonging to one of 23 novel Vbeta groups
CASSV

SEQ ID NO: 17 - protein, 5 aa, Artificial sequence
  A carboxy terminal portion of a Vbeta-segment, belonging to one of 23 novel Vbeta groups
CASSI

SEQ ID NO: 18 - protein, 5 aa, Artificial sequence
  A carboxy terminal portion of a Vbeta-segment, belonging to one of 23 novel Vbeta groups
CASSD

SEQ ID NO: 19 - protein, 5 aa, Artificial sequence
  A carboxy terminal portion of a Vbeta-segment, belonging to one of 23 novel Vbeta groups
CASSP

SEQ ID NO: 20 - protein, 5 aa, Artificial sequence
  A carboxy terminal portion of a Vbeta-segment, belonging to one of 23 novel Vbeta groups
CASGL

SEQ ID NO: 21 - protein, 5 aa, Artificial sequence
  A carboxy terminal portion of a Vbeta-segment, belonging to one of 23 novel Vbeta groups
CATSR

SEQ ID NO: 22 - protein, 5 aa, Artificial sequence
  A carboxy terminal portion of a Vbeta-segment, belonging to one of 23 novel Vbeta groups
CAISE

SEQ ID NO: 23 - protein, 6 aa, Artificial sequence
  A carboxy terminal portion of a Vbeta-segment, belonging to one of 23 novel Vbeta groups
CATSDL

SEQ ID NO: 24 - DNA, 12 bp, Artificial sequence
  Consensus DNA sequence encoding carboxy terminal portion of Vbeta segments belonging to a novel group
gtgtacttctgt

SEQ ID NO: 25 - DNA, 12 bp, Artificial sequence
  Consensus DNA sequence encoding carboxy terminal portion of Vbeta segments belonging to a novel group
tatttctgtgcc

SEQ ID NO: 26 - DNA, 12 bp, Artificial sequence
  Consensus DNA sequence encoding carboxy terminal portion of Vbeta segments belonging to a novel group
tatctctgcagc

SEQ ID NO: 27 - DNA, 12 bp, Artificial sequence
  Consensus DNA sequence encoding carboxy terminal portion of Vbeta segments belonging to a novel group
ytytgygccagc

SEQ ID NO: 28 - DNA, 12 bp, Artificial sequence
  Consensus DNA sequence encoding carboxy terminal portion of Vbeta segments belonging to a novel group
ctctgtgcctgg

SEQ ID NO: 29 - DNA, 12 bp, Artificial sequence
  Consensus DNA sequence encoding carboxy terminal portion of Vbeta segments belonging to a novel group
atctgcagtgct

SEQ ID NO: 30 - DNA, 12 bp, Artificial sequence
  Consensus DNA sequence encoding carboxy terminal portion of Vbeta segments belonging to a novel group
tgygccagyagy

SEQ ID NO: 31 - DNA, 12 bp, Artificial sequence
  Consensus DNA sequence encoding carboxy terminal portion of Vbeta segments belonging to a novel group
tgtgccaccagc

SEQ ID NO: 32 - DNA, 12 bp, Artificial sequence
  Consensus DNA sequence encoding carboxy terminal portion of Vbeta segments belonging to a novel group
tgtgcctggagt

SEQ ID NO: 33 - DNA, 12 bp, Artificial sequence
  Consensus DNA sequence encoding carboxy terminal portion of Vbeta segments belonging to a novel group
tgcagcgttgaa

SEQ ID NO: 34 - DNA, 12 bp, Artificial sequence
  Consensus DNA sequence encoding carboxy terminal portion of Vbeta segments belonging to a novel group
tgcagtgctaga

SEQ ID NO: 35 - DNA, 15 bp, Artificial sequence
  Consensus DNA sequence encoding carboxy terminal portion of Vbeta segments belonging to a novel group
tgygccagcagyttr

SEQ ID NO: 36 - DNA, 15 bp, Artificial sequence
  Consensus DNA sequence encoding carboxy terminal portion of Vbeta segments belonging to a novel group
tgygccagcagccaa

SEQ ID NO: 37 - DNA, 15 bp, Artificial sequence
  Consensus DNA sequence encoding carboxy terminal portion of Vbeta segments belonging to a novel group
tgtgccagcagttay

SEQ ID NO: 38 - DNA, 15 bp, Artificial sequence
  Consensus DNA sequence encoding carboxy terminal portion of Vbeta segments belonging to a novel group
tgtgccagcagtgar

SEQ ID NO: 39 - DNA, 15 bp, Artificial sequence
  Consensus DNA sequence encoding carboxy terminal portion of Vbeta segments belonging to a novel group
tgtgccagcagcgta

SEQ ID NO: 40 - DNA, 15 bp, Artificial sequence
  Consensus DNA sequence encoding carboxy terminal portion of Vbeta segments belonging to a novel group
tgtgccagtagtata

SEQ ID NO: 41 - DNA, 15 bp, Artificial sequence
  Consensus DNA sequence encoding carboxy terminal portion of Vbeta segments belonging to a novel group
tgtgccagcagtgac

SEQ ID NO: 42 - DNA, 15 bp, Artificial sequence
  Consensus DNA sequence encoding carboxy terminal portion of Vbeta segments belonging to a novel group
tgtgccagctcacca

SEQ ID NO: 43 - DNA, 15 bp, Artificial sequence
  Consensus DNA sequence encoding carboxy terminal portion of Vbeta segments belonging to a novel group
tgtgctagtggtttg

SEQ ID NO: 44 - DNA, 15 bp, Artificial sequence
  Consensus DNA sequence encoding carboxy terminal portion of Vbeta segments belonging to a novel group
tgtgccaccagcaga

SEQ ID NO: 45 - DNA, 15 bp, Artificial sequence
  Consensus DNA sequence encoding carboxy terminal portion of Vbeta segments belonging to a novel group
tgtgccatcagtgag

SEQ ID NO: 46 - DNA, 18 bp, Artificial sequence
  Consensus DNA sequence encoding carboxy terminal portion of Vbeta segments belonging to a novel group
tgtgccaccagtgatttg

SEQ ID NO: 47 - DNA, 12 bp, Artificial sequence
  DNA consensus sequence which includes all known sequences encoding a Dbeta segment, and one of 13 sequences each of which encoding a CDR3 specific portion of one of the 13 Jbeta segments
gggacwrgsggs

SEQ ID NO: 48 - DNA, 12 bp, Artificial sequence
  DNA sequence of CDR3 encoding N-terminal portion of Jbeta segment
actgaagctttc

SEQ ID NO: 49 - DNA, 12 bp, Artificial sequence
  DNA sequence of CDR3 encoding N-terminal portion of Jbeta segment
tatggctacacc

SEQ ID NO: 50 - DNA, 15 bp, Artificial sequence
  DNA sequence of CDR3 encoding N-terminal portion of Jbeta segment
ggaaacaccatatat

SEQ ID NO: 51 - DNA, 15 bp, Artificial sequence
  DNA sequence of CDR3 encoding N-terminal portion of Jbeta segment
aatgaaaaactgttt

SEQ ID NO: 52 - DNA, 15 bp, Artificial sequence
  DNA sequence of CDR3 encoding N-terminal portion of Jbeta segment
aatcagccccagcat

SEQ ID NO: 53 - DNA, 18 bp, Artificial sequence
  DNA sequence of CDR3 encoding N-terminal portion of Jbeta segment
tataattcacccctccac

SEQ ID NO: 54 - DNA, 15 bp, Artificial sequence
  DNA sequence of CDR3 encoding N-terminal portion of Jbeta segment
tacaatgagcagttc

SEQ ID NO: 55 - DNA, 15 bp, Artificial sequence
  DNA sequence of CDR3 encoding N-terminal portion of Jbeta segment
accggggagctgttt

SEQ ID NO: 56 - DNA, 15 bp, Artificial sequence
  DNA sequence of CDR3 encoding N-terminal portion of Jbeta segment
acagatacgcagtat

SEQ ID NO: 57 - DNA, 15 bp, Artificial sequence
  DNA sequence of CDR3 encoding N-terminal portion of Jbeta segment
aaaaacattcagtac

SEQ ID NO: 58 - DNA, 12 bp, Artificial sequence
  DNA sequence of CDR3 encoding N-terminal portion of Jbeta segment
gagacccagtac

SEQ ID NO: 59 - DNA, 18 bp, Artificial sequence
  DNA sequence of CDR3 encoding N-terminal portion of Jbeta segment
ggggccaacgtcctgact

SEQ ID NO: 60 - DNA, 12 bp, Artificial sequence
  DNA sequence of CDR3 encoding N-terminal portion of Jbeta segment
tacgagcagtac

SEQ ID NO: 61 - DNA, 36 bp, Artificial sequence
  A degenerate probe having a Vbeta specific module, specific for Vbeta-segments belonging to group No. 1
gtgtacttctgtgggacwrgsggsactgaagctttc

SEQ ID NO: 62 - DNA, 36 bp, Artificial sequence
  A degenerate probe having a Vbeta specific module, specific for Vbeta-segments belonging to group No. 1
gtgtacttctgtgggacwrgsggstatggctacacc

SEQ ID NO: 63 - DNA, 39 bp, Artificial sequence
  A degenerate probe having a Vbeta specific module, specific for Vbeta-segments belonging to group No. 1
gtgtacttctgtgggacwrgsggsggaaacaccatatat

SEQ ID NO: 64 - DNA, 39 bp, Artificial sequence
  A degenerate probe having a Vbeta specific module, specific for Vbeta-segments belonging to group No. 1
gtgtacttctgtgggacwrgsggsaatgaaaaactgttt

SEQ ID NO: 65 - DNA, 39 bp, Artificial sequence
  A degenerate probe having a Vbeta specific module, specific for Vbeta-segments belonging to group No. 1
gtgtacttctgtgggacwrgsggsaatcagccccagcat

SEQ ID NO: 66 - DNA, 42 bp, Artificial sequence
  A degenerate probe having a Vbeta specific module, specific for Vbeta-segments belonging to group No. 1
gtgtacttctgtgggacwrgsggstataattcacccctccac

SEQ ID NO: 67 - DNA, 39 bp, Artificial sequence
  A degenerate probe having a Vbeta specific module, specific for Vbeta-segments belonging to group No. 1
gtgtacttctgtgggacwrgsggstacaatgagcagttc

SEQ ID NO: 68 - DNA, 39 bp, Artificial sequence
  A degenerate probe having a Vbeta specific module, specific for Vbeta-segments belonging to group No. 1
gtgtacttctgtgggacwrgsggsaccggggagctgttt

SEQ ID NO: 69 - DNA, 39 bp, Artificial sequence
  A degenerate probe having a Vbeta specific module, specific for Vbeta-segments belonging to group No. 1
gtgtacttctgtgggacwrgsggsacagatacgcagtat

SEQ ID NO: 70 - DNA, 39 bp, Artificial sequence
  A degenerate probe having a Vbeta specific module, specific for Vbeta-segments belonging to group No. 1
gtgtacttctgtgggacwrgsggsaaaaacattcagtac

SEQ ID NO: 71 - DNA, 36 bp, Artificial sequence
  A degenerate probe having a Vbeta specific module, specific for Vbeta-segments belonging to group No. 1
gtgtacttctgtgggacwrgsggsgagacccagtac

SEQ ID NO: 72 - DNA, 42 bp, Artificial sequence
  A degenerate probe having a Vbeta specific module, specific for Vbeta-segments belonging to group No. 1
gtgtacttctgtgggacwrgsggsggggccaacgtcctgact

SEQ ID NO: 73 - DNA, 36 bp, Artificial sequence
  A degenerate probe having a Vbeta specific module, specific for Vbeta-segments belonging to group No. 1
gtgtacttctgtgggacwrgsggstacgagcagtac

SEQ ID NO: 74 - DNA, 29 bp, Artificial sequence
  Single strand DNA oligonucleotide
tgcttctgatggctcaaacacagcgacct

(19) United States
(12) Patent Application Publication
(10) Pub. No.: US 2007/0238099 A1
(43) Pub. Date: Oct. 11, 2007

(54) Antigen Receptor Variable Region Typing

(76) Inventors: Irun R. Cohen, Rechovot (IL); Daniel Douek, Bethesda, MD (US); Avishai Mimran, Rehovot (IL); Pnina Carmi, Rehovot (IL); Francisco Javier Quintana, Capital Federal (AR)

Correspondence Address: Martin D. Moynihan;PRTSI, Inc., P.O. Box 16446, Arlington, VA, 22215, US

(21) Appl. No.: 10/581,913

(22) PCT Filed: Oct. 25, 2004
(86) PCT No.: PCT/IL04/00972

§ 371 Date: Feb. 15, 2007

Related U.S. Application Data
(60) Provisional application No. 60/527,273, filed on Dec. 8, 2003.

(51) Int. Cl. C12Q 1/68; C07H 21/02; C07H 21/04; C12M 1/00

(52) U.S. Cl. 435/6; 435/287.2; 536/24.3

(57) ABSTRACT

A method of typing a variable region of a specific variant of an antigen receptor chain is disclosed. The method comprises: (a) exposing a probe set to a sense or antisense strand of a polynucleotide encoding at least a portion of the variable region of the specific variant of the antigen receptor chain, wherein the probe set includes a plurality of probe molecules, wherein each probe molecule of the plurality of probe molecules is substantially complementary to a sense or antisense strand of a nucleic acid sequence region of a specific polynucleotide encoding a variant of the antigen receptor chain, the nucleic acid sequence region encoding a specific combination of at least two variable region segments of the antigen receptor chain; and (b) measuring a hybridization of each probe molecule of the plurality of probe molecules with the sense or antisense strand of the nucleic acid sequence region of the polynucleotide encoding at least a portion of the variable region of the specific variant of the antigen receptor chain, thereby typing the variable region of the specific variant of the antigen receptor chain.

### FIELD AND BACKGROUND OF THE INVENTION

The present invention relates to methods of typing variable regions of antigen receptor chains, to probe arrays for practicing such typing, and to probe sets for generating such arrays. More particularly, the present invention relates to methods of typing variable region segment combinations of T-cell receptor (TCR) chains encoded by polynucleotides or antisense sequences thereof, to polynucleotide probe arrays for practicing such typing, and to polynucleotide probe sets for generating such arrays.

Diseases associated with a protective or pathogenic antigen specific immune response, such as infectious, autoimmune, allergic, transplantation related, malignant, and inflammatory diseases, include numerous highly debilitating and/or lethal diseases whose medical management is suboptimal, for example, with respect to prevention, diagnosis, treatment, patient monitoring, prognosis, and/or drug design.

For example, dangerous infectious diseases for which no optimal medical management methods are available include acquired immunodeficiency syndrome (AIDS) caused by human immunodeficiency virus (HIV), influenza, malaria, hepatitis, tuberculosis, cholera, Ebola virus infection, and severe acute respiratory syndrome (SARS). Such diseases are collectively responsible for millions of deaths worldwide each year. Ominously, diseases such as AIDS, and antibiotic-resistant bacterial and mycobacterial infections, such as antibiotic-resistant staphylococcal and tuberculosis infections, respectively, to which there are no satisfactory cures for most affected individuals, are on the increase. Also of concern is the widely perceived and anticipated threat of biological warfare using agents causing lethal infectious diseases, such as anthrax, smallpox, and bubonic plague. Hence, society is confronted with the challenge of vaccinating on relatively short notice large numbers of persons against such pathogens. Infectious diseases require better diagnostic discrimination between persons who will be susceptible to a particular vaccination and persons who will not respond. Certain infections can trigger autoimmune responses, and it is important to be able to diagnose persons who are destined to develop autoimmune diseases. With respect to vaccination strategies against infectious diseases, significant numbers of people have various degrees of immune malfunction—genetic, drug-induced, or acquired by infection or neoplasia—that could lead to serious complications upon exposure to live vaccines such as vaccinia. Idiosyncratic reactions to killed virus or viral subunit vaccines could also cause serious illness. Therefore, it has become essential to develop ways to survey the immune state of large numbers of people in a manner that is fast, reliable, safe and relatively inexpensive. In particular, it is necessary to be able to stratify individuals so as to predict the potential hazards of various vaccinations.

Autoimmune diseases represent a large group of highly debilitating and/or lethal diseases which includes such widespread and devastating diseases as rheumatoid arthritis, type I diabetes and multiple sclerosis. Traditionally, immunologic diagnosis and prognosis has been based on an attempt to correlate each condition with a specific immune reactivity, such as an antibody or a T-lymphocyte response to a single antigen specific for the disease entity. This approach has been largely unsuccessful for various reasons, such as the absence of specific antigens serving as markers of the disease. In the case of autoimmune diseases, this approach has been unsuccessful due to, for example, immunity to multiple self-antigens, as exemplified by type I diabetes which may be associated with a dozen different antigens, and due to the fact that a significant number of healthy persons may manifest antibodies or T-lymphocyte reactivities to self-antigens targeted in autoimmune diseases, such as insulin, DNA, myelin basic protein, thyroglobulin and others. For this reason, false positive tests are not uncommon. Hence, there is a real danger of making a false diagnosis based on the determination of a given immune reactivity. Novel approaches, therefore, are needed to support the diagnoses of specific immune conditions in a way that would justify specific therapeutic decisions.

Malignant diseases such as breast cancer, lung cancer, colorectal cancer, melanoma and prostate cancer are a tremendous medical and economic burden, particularly in industrialized populations. The immunotherapy of cancer is a situation in which it would be advantageous to classify persons with different types of immune reactivities to self-antigens; many, if not most, tumor-associated antigens are self-antigens. Thus, it is important in the design of therapeutic tumor vaccines to know what kind of immune potential is present in the patient. In individuals who have received chemotherapy and stem cell transplants for leukemias and other cancers, the monitoring of the overall breadth of the recovering immune system becomes crucially important. An immune system with a broader repertoire reflects one with more potential to combat infections.

Transplantation related diseases such as graft rejection and graft-versus-host disease are major causes of failure of therapeutic transplantation, a medical procedure of last resort broadly practiced for treating numerous life-threatening diseases, such as cardiac, renal, pulmonary, hepatic and pancreatic failure.

Allergic diseases, such as allergy to seasonal pollens, ragweed, dust mites, pet fur, cosmetics, and various foods are significantly debilitating to a large proportion of the population, can be fatal, and are of great economic significance due to the large market for allergy drugs.

The need for optimal methods of monitoring immune responses and disease progression is acutely felt in the pharmaceutical industry in the development of new therapeutic biological agents and drugs. Autoimmune and degenerative diseases are intrinsically difficult to deal with pharmaceutically. Not only are these diseases chronic, but the individual patients enrolled in treatment trials tend to be in different states of responsiveness. Thus it is difficult to devise a single dose of a drug and a treatment schedule that will be optimal for each individual. Some individuals need larger or smaller doses, or more or less frequent administration for an optimal response. Thus it is all too easy to miss the mark, and even effective drugs have failed to reach statistical significance in trials. Indeed, it is costly and hazardous to risk the success of a new drug on a long-term trial of one or a few doses or modes of administration. The industry critically requires predictive markers to stratify individuals and design trials based providing critical immunologic information regarding the response of the test individuals. Clinical trials of anti-inflammatory drugs have focused on the disease as the only endpoint, and have failed to monitor the cause of the disease. Hence, methods of characterizing antigenic specificities of the immune system could provide the information needed to optimize effectiveness and save time in arriving at dosing and other variables.

Hence, there is an urgent need for novel and improved methods for facilitating optimal performance of various aspects of medical management of a vast range of antigen associated diseases.

The adaptive immune system normally functions to afford rapid, specific and dynamic responses to a huge variety of antigen specific insults, in particular invasion by microbial pathogens and non-self cells. By virtue of B- and T-lymphocytes being the antigen specific effectors of humoral and cellular immunity, respectively, these cell types play a central role in the body's defense against antigen-associated diseases. The antigenic specificity of B- and T-lymphocyte mediated immune responses is conferred by B-cell receptors (BCRs) and TCRs, antigen specific receptors clonally distributed on individual lymphocytes, whose repertoire of antigenic specificity is generated via somatic gene rearrangement. B-cell receptors and TCRs are bound to the membrane of B- and T-lymphocytes together with coreceptors, which mediate specific signals following ligand recognition. In the case of B-lymphocytes, in order to effect humoral immune responses, such cells additionally secrete soluble BCRs in the form of antigen specific antibodies. While the function of lymphocytes is normally protective, under conditions of immune dysregulation B- and T-lymphocytes may mediate antigen specific immunity resulting in disease pathogenesis, either as a result of misdirected immunity, as in the case of autoimmune, allergic, transplantation-related and inflammatory diseases; or as a result of insufficient immunity, as in the case of infectious, and malignant diseases.

T-lymphocytes play a critical role in immune responses against infectious agents and in the body's natural defenses against neoplastic diseases. A typical T-lymphocyte mediated immune response is characterized by recognition of a particular antigen, secretion of growth-promoting cytokines, and proliferative expansion to provide additional T-cells to recognize and eliminate the foreign antigen. There are two major T-lymphocyte types, helper T-lymphocytes and cytotoxic T lymphocytes (CTLs). The normal function of helper T-lymphocytes is to secrete cytokines such as IL-2 which promote activation and proliferation of antigen specific B- and T-lymphocytes, and that of CTLs is to trigger apoptotic death of self or allogeneic cells containing intracellular antigens recognized as foreign by the immune system. Hence, T-lymphocyte effector functions are activated in response to self cells containing intracellular antigens such as pathogen derived antigens, tumor-associated antigens (TAAs), self-antigens in the case of autoimmune disease, or graft or host cells displaying allogeneic/xenogeneic antigens relative to host or graft T-lymphocytes in the case of graft rejection or GVHD, respectively (Krensky A. et al., 1990. N Engl J Med. 322:510).

T-cell receptors are composed of a heterodimer of transmembrane molecules, with about 95% of TCRs being composed of an αβ dimer and the remainder of a γδ dimer. T-cell receptor α, β, γ and δ chains comprise a transmembrane constant region and a variable region in the extracellular domain, similarly to immunoglobulins (Ig's). Signal transduction of TCRs is transmitted via CD3/ζζ complex, an associated multi-subunit signaling complex comprising signal transducing subunits. Unlike antibodies, TCRs do not recognize native antigens but rather a complex of an intracellularly processed polypeptide or lipid antigen fragment “presented” at the surface of self cells by a specialized antigen-presenting molecule (APM); MHC in the case of peptide antigens and CD1 in the case of lipid antigens. The two main types of MHC molecules, MHC class I and MHC class II, serve distinct functions in T-lymphocyte mediated immunity and in accordance are expressed on distinct cells types. Major histocompatibility complex class I molecules are expressed on the surface of virtually all cells in the body while MHC class II molecules are expressed on a restricted subset of specialized antigen-presenting cells (APCs) involved in T-lymphocyte maturation and priming, such as dendritic cells and macrophages. Major histocompatibility complex class I and II molecules respectively specifically present antigen to either CTLs or helper T-lymphocytes which specifically display CD8 and CD4 MHC coreceptors, respectively, enabling such specific engagement. Similarly to MHC class II, CD1 is mainly expressed on professional APCs.

B-lymphocyte mediated immune responses are initially mediated by specific recognition and binding of antigen by membranal BCRs (IgM and IgD isotype) which as a consequence is endocytosed, processed, and displayed at the cell surface by MHC class II molecules so as to enable activation of helper T-lymphocytes. Other antigen presenting cells, such as dendritic cells or macrophages, can also activate helper T-cells. Such activated helper T-lymphocytes in turn engage and stimulate B-lymphocytes by releasing cytokines such as IL-4 to induce their differentiation into plasma cells which secrete large quantities of antibodies. Antibodies mediate humoral immunity by specifically binding to foreign antigenic determinants on the surface of pathogens such as viruses, parasites, and bacteria, leading to their neutralization and elimination from the body via activation of the complement cascade culminating in oxidative burst killing of pathogen, and via Fc receptor mediated phagocytotic clearing of pathogen. Furthermore, the complement cascade generates complement protein cleavage products functioning as opsonins having the capacity to trigger non-specific inflammatory responses involving accumulation of phagocytes such as neutrophils and macrophages at sites of infection, thereby further sensitizing the immune system against the foreign antigen.

The most fundamental mechanism whereby the great variability of antigen receptor specificity is generated is via combinatorial rearrangement of variable region gene segments of antigen receptor gene loci (for review, refer, for example, to Janeway, C A. et al., “Immunobiology”, 5th ed. Garland Publishing, New York and London, c2001). This rearrangement process, commonly known as “V(D)J” recombination, which occurs during lymphocyte maturation, has the capacity to generate an antigen receptor repertoire which is orders of magnitude greater than the total number of lymphocytes present in an organism at any one time. Similarly to Ig heavy and light chains, TCR α and β chains include an amino-terminal variable (V) region and a carboxy terminal constant (C) region. The gene segment organization in TCR chains is generally homologous to that of the Ig gene segments whereby the TCRα locus comprises Vα and Jα gene segments, similarly to the Ig light chains, and the TCRβ, locus comprises Dβ gene segments as well as Vβ and Jβ gene segments, similarly to Ig heavy-chain. T-cell receptor loci have a similar number of V gene segments but a greater number of J gene segments than Ig loci, and display greater variability between gene segment junctions during gene rearrangement.

The genomic organization of the variable gene segments of the human TCRβ locus involves an upstream cluster of Vβ-segment genes followed by two segment gene clusters each encoding a Dβ-segment, multiple Jβ-segments, and a Cβ-segment. To date about 60 different Vβ-segments (see Table 1, below), 2 different Dβ-segments and 13 different Jβ-segments have been identified. Not all Vβ-segment genes have been identified, however, with certain Vβ-segment genes being optionally expressed and others, termed pseudogenes, never being expressed. Taking codon usage variability as well as allelic variation into account there were as of October 2004 about 128 known distinct genetic sequences encoding Vβ-segments (refer, for example, to http://imgt.cines.fr:8104/textes/IMGTrepertoire/Proteins/taballeles/human/TRB/TRBV/Hu_TRBVall.html; or http://imgt.cines.fr:8104/textes/IMGT repertoire/Proteins/alleles/human/HuAl_list.html#trbv).

The variable regions of antigen receptor chains comprise three hypervariable loop structures referred to as complementarity determining regions (CDRs). In TCR chains, the first (CDR1) and second (CDR2) CDR loops are comprised within the Vβ-segment and contact the relatively less variable MHC component of the MHC:antigen complex. In contrast, the third CDR loop (CDR3), which is responsible for making the contact with the presented antigen, is the most highly variable, being formed by the carboxy terminal portion of the Vβ-segment, the entire Dβ-segment and the amino terminal end of the Jβ-segment.

The antigenic specificity of any given antigen receptor is therefore largely dictated by the particular combination of rearranged gene segments with which it is composed, and at the whole organism level, the repertoire of actual and potential antigenic specificities of an antigen receptor in a specific individual will similarly be largely dictated by the particular repertoire of antigen receptor gene segment combinations represented in the individual.

The repertoire of rearrangements of an antigen receptor chain in an individual is thus driven by two principal types of processes. The first type of process, which occurs during lymphocyte maturation, comprises an initial rearrangement phase involving generation of a repertoire of an antigen receptor chain in which the particular variable segment alleles encoded by the germline of the individual are randomly rearranged. In a subsequent negative selection phase, lymphocytes expressing potentially autoreactive antigen receptors are eliminated. The second type of process, driven by antigen specific immune responses, involves clonal expansion and memory cell differentiation of lymphocytes expressing antigen receptors optimally binding the antigens targeted by such immune responses.

Thus, the capacity to optimally type the specificity repertoire of an antigen receptor of an individual could be used to optimally characterize the types of antigens, pathogens and associated diseases encountered in the individual's lifetime, the types of immune responses the individual has mounted in response to such antigens, pathogens and associated diseases, and the potential capacity of the individual's immune system to react in the future against specific antigens, pathogens and associated diseases. Such typing capacity could be used to optimally identify in the antigen receptor specificity repertoire of the individual a known specificity pattern correlating with an immunological phenotype associated with the disease. Such identification could then be used to optimally categorize the individual with respect to the disease phenotype and hence could be used to optimize medical management of the disease in the individual. Such a typing method could further be used to identify novel specificity patterns across groups of individuals sharing an immunological phenotype characteristic of an antigen associated disease. Such phenotypes would include, for example, histories, states, courses, susceptibilities, and therapeutic responses associated with an antigen associated disease.

Therefore, the capacity to optimally type a repertoire of specificities of an antigen receptor chain could be used for facilitating optimal medical management of antigen associated diseases, including infectious, malignant, autoimmune, transplantation related, allergic, malignant and inflammatory diseases. Particular aspects of medical management which could be optimized as a result of optimal typing of an antigen receptor specificity repertoire of an individual would notably include prevention, diagnosis, treatment, patient monitoring, prognosis, drug design, and the like.

Various prior art approaches have been suggested or attempted for typing antigen receptor specificity repertoires.

One approach involves typing an antigen receptor specificity repertoire indirectly as a function of CDR length repertoire. One example of such an approach is the “spectratyping” technique (reviewed in Janeway, Charles A. et al. “Immunobiology”, 5th ed. Garland Publishing, New York and London, c2001), a PCR based method that separates genetic sequences encoding antigen receptors on the basis of CDR3 length using primers specific for individual V gene segments at one end, and specific for a conserved part of the C region at the other end so as to generate a set of amplification fragments spanning the CDR3. This generates a set of PCR products having lengths representing the CDR3 length repertoire such that changes in length distribution can be correlated to changes in the antigen receptor repertoire. Another approach based on typing a CDR length repertoire is the “immunoscope” technique (reviewed in Ria F. et al., 2001. Curr Mol Med. 1:297-304), an RT-PCR based approach in which a bulk lymphocyte population is separated into hundreds to thousands of groups based on rearranged antigen receptor V/J gene segments and the resulting length of the CDR3 so as to attempt to track clonal shifts. A further approach based on typing a CDR length repertoire comprises using RT-PCR to amplify mRNA transcripts from a cell sample using family-specific Vβ oligonucleotide primers, and analyzing the cDNA products on a DNA sequencer to visualize the ranges of CDR3 lengths (Cottrez et al., 1994. J Immunol Methods 172: 85-94; see also Gorski et al., 1994. J Immunol. 152:5109-5119).

Another approach involves cloning Ig chains, and expressing them in genetically transformed host cells for analysis thereof. One example of such an approach involves cloning antibodies from diseased human tissues, expressing them in eukaryotic cell lines, and analyzing them via immunoT cytochemistry and FACS analysis (Williamson et al., 2001. Proc Natl Acad Sci USA. 98:1793-8). Another example of such an approach involves establishing B-cell hybridomas from human individuals and characterizing their Ig specificity repertoires by ELISA and sequencing (Baxendale et al., 2000. Eur J Immunol. 30:1214-23).

Further approaches involve utilizing random sequencing or RNase protection assays (Okada et al., 1989. J Exp Med. 169:1703-1719; Singer et al., 1990. EMBO J. 9:3641-3648), TCR mini-libraries in E. coli generated by anchored or inverse PCR (Rieux-Laucat et al., 1993. Eur J Immunol. 23:928-934; Uematsu et al., 1991. Immunogenetics 34:174-178), and variable region gene usage analysis using available specific monoclonal antibodies (mAb's; Genevee et al., 1994. Int Immunol. 6:1497-1504).

Yet a further approach involves PCR amplifying cDNA derived from cell samples via PCR using family-specific Vα and Vβ oligonucleotide primers, and analyzing the PCR reaction products by Southern blotting using α-chain or β-chain constant region gene probes to detect a specific TCR Vα or Vβ family (Oaks et al., 1995. Am J Med Sci. 309:26-34).

Still a further approach involves PCR amplifying cDNA from cell samples using family-specific Vβ oligonucleotide primers, and analyzing the PCR reaction products using the “single-strand conformation polymorphism” (SSCP) technique, wherein the PCR reaction products are separated into single strands and electrophoresed on a non-denaturing polyacrylamide gel, such that DNA fragments having the same length are made further separable by differences in secondary structure. Using this method, the amplified DNA from polyclonal lymphocytes is visualized as a “smear” comprising discrete bands being indicative of T-cell clonal expansion (European Patent Application No. 0653 493 A1, filed 30 Apr. 1993).

An additional approach involves determining VDJ junction size patterns in twenty-four human Vβ subfamilies by PCR amplifying cDNA from malignant tissue biopsies using Vβ family-specific primers, and sequencing the resultant PCR products. To “refine” the T-cell repertoire analysis, a second set of Vβ family-specific PCR reactions of interest are further subjected to primer extension “run-off” reactions using a fluorophore labeled Cβ primer and/or using one of thirteen Jβ-family specific, fluorophore-labeled Jβ primers. The run-off reaction products are then analyzed on additional sequencing gels (Puisieux et al., 1994. J Immunol. 153:2807-18). A refinement of this approach involves using twenty-five Vβ family-specific PCR amplifications, twenty-five Cβ run-off reactions, and 325 Jβ run-off reactions (25Vβ×13Jβ=325). Each run-off reaction is analyzed by electrophoresing an aliquot on a polyacrylamide gel (Pannetier et al., 1995. Immunol Today 16:176-181).

Yet additional approaches are the “intrafamily” and “interfamily” fragment PCR analysis approaches (PCT Patent Application No. WO 97/18330 to Dau et al.). In the interfamily approach the PCR is used for amplifying cDNA from cell samples using primers specific for each Vβ family, and quantitatively comparing reaction products. This approach is disadvantageous in that it involves a requirement for optimization of reaction conditions necessary for optimizing primer efficiencies and to stop all reactions in log phase for all Vβ families. In the intrafamily approach fragments generated by a single Vβ primer are compared to avoid the interfamily analysis optimization requirements.

Still an additional approach involves utilizing antigen microarrays wherein standard gene spotting technology is used to spot arrays of large numbers of antigens on glass slides. Small amounts of test sera are applied to the coated slides, and antibodies binding to these molecules are detected via laser scanning (WIPO Publication No. WO0208755).

Yet still an additional approach has suggested employing DNA oligonucleotide probe microarrays for typing individual variable region segments (WIPO Publication No. WO03044225A2).

However, all prior art approaches for typing antigen receptor specificity repertoires, are highly suboptimal. Approaches involving typing of an antigen receptor specificity repertoire via typing of a CDR/VDJ junction length repertoire, are essentially non informative with respect to characterizing antigen receptor specificities per se. Approaches involving genetic transformation of host cells to express antigen receptor polypeptides, nucleic acid sequencing, antigen receptor cognate antigen probes, antibody probes specific for antigen receptor variants, and electrophoresis are highly labor intensive, extremely restricted in the scope of specificities which may be typed and/or are unsuitable for high throughput analysis. Approaches based on antigen microarrays are highly impractical for typing specificity repertoires of TCRs since TCRs, unlike antibodies, do not recognize native antigen molecules, but processed antigen fragments complexed with specific MHC molecules, which are impossible to generate on the repertoire scale. Furthermore, since T-lymphocytes do not secrete their TCR molecules, it is highly impractical to obtain large numbers of free TCR molecules.

Critically, no prior art approach enables high throughput typing of antigen receptor specificity repertoires as a function of rearranged variable region segment combinations.

Thus, all prior art approaches have failed to provide an adequate solution for typing antigen receptor specificity repertoires.

There is thus a widely recognized need for, and it would be highly advantageous to have, a method devoid of the above limitation.

### SUMMARY OF THE INVENTION

According to one aspect of the present invention there is provided a method of typing a variable region of a specific variant of an antigen receptor chain, the method comprising: (a) exposing a probe set to a sense or antisense strand of a polynucleotide encoding at least a portion of the variable region of the specific variant of the antigen receptor chain, wherein the probe set includes a plurality of probe molecules, wherein each probe molecule of the plurality of probe molecules is substantially complementary to a sense or antisense strand of a nucleic acid sequence region of a specific polynucleotide encoding a variant of the antigen receptor chain, the nucleic acid sequence region distinctly encoding a specific combination of at least two variable region segments of the antigen receptor chain; and (b) measuring a hybridization of the each probe molecule of the plurality of probe molecules with the sense or antisense strand of the nucleic acid sequence region of the polynucleotide encoding at least a portion of the variable region of the specific variant of the antigen receptor chain, thereby typing the variable region of the specific variant of the antigen receptor chain.

According to further features in preferred embodiments of the invention described below, each distinct probe molecule of the probe set is attached to a probe array at a specific addressable location included in the probe array.

According to still further features in the described preferred embodiments, step (b) is effected by measuring a collective hybridization of the sense or antisense strand of the polynucleotide encoding at least the portion of the variable region of the specific variant of the antigen receptor chain, with each distinct probe molecule of each distinct subset of probe molecules of a group of distinct subsets of probe molecules of the probe set, wherein the group of distinct subsets of probe molecules includes a number of distinct subsets of probe molecules selected from a range of 1-299 distinct subsets of probe molecules.

According to still further features in the described preferred embodiments, each distinct subset of probe molecules of the group of distinct subsets of probe molecules includes a number of distinct probe molecules selected from a range of 1-128 distinct probe molecules.

According to still further features in the described preferred embodiments, each distinct subset of probe molecules of the group of distinct subsets of probe molecules is attached to a probe array at a specific addressable location of a plurality of addressable locations included in the probe array.

According to still further features in the described preferred embodiments, the polynucleotide encoding at least the portion of the variable region of the specific variant of the antigen receptor chain is a complementary DNA molecule.

According to another aspect of the present invention there is provided a probe array comprising a support including a plurality of addressable locations and a probe set including a plurality of probe molecules, wherein each probe molecule of the plurality of probe molecules is attached to a specific addressable location of the plurality of addressable locations of the support, and is substantially complementary to a sense or antisense strand of a nucleic acid sequence region of a specific polynucleotide encoding a variant of an antigen receptor chain, the nucleic acid sequence region distinctly encoding a specific combination of at least two variable region segments of the antigen receptor chain.

According to still further features in the described preferred embodiments, the probe array includes the plurality of addressable locations at a surface density of at least 625 specific addressable locations per square centimeter of a support comprised in the probe array.

According to yet another aspect of the present invention there is provided a probe set comprising a plurality of probe molecules, each probe molecule of the plurality of probe molecules being substantially complementary to a sense or antisense strand of a nucleic acid sequence region of a specific polynucleotide encoding a variant of an antigen receptor chain, the nucleic acid sequence region distinctly encoding a specific combination of at least two variable region segments of the antigen receptor chain.

According to further features in preferred embodiments of the invention described below, the probe set includes a number of probe molecules selected from the group consisting of 2, 3, 4, 5, 6, 7, 8, 9, 10, 11, 12, 13, 14, 15, 16, 17, 18, 19, 20, 21, 22, 23, 24 or 25 probe molecules.

According to still further features in the described preferred embodiments, the probe set includes a number of probe molecules selected from a range of 26-30, 31-35, 36-40, 41-45, 46-50, 51-55, 56-60, 61-65, 66-70, 71-75, 76-80, 81-85, 86-90, 91-95, 96-100, 101-150, 151-200, 201-250, 251-300, 301-350, 351-400, 401-450, 451-500, 501-550, 551-600, 601-650, 651-700, 701-750, 751-800, 801-850, 851-900, 901-950, 951-1,000, 1,001-1,100, 1,101-1,200, 1,201-1,300, 1,301-1,400, 1,401-1,500, 1,501-1,600, 1,601-1,700, 1,701-1,800, 1,801-1,900, 1,901-2,000, 2,001-2,100, 2,101-2,200, 2,201-2,300, 2,301-2,400, 2,401-2,500, 2,501-2,600, 2,601-2,700, 2,701-2,800, 2,801-2,900, 2901-3000, 3,001-3,500, 3,501-4,000, 4,001-4,500, 4,501-5,000, 5,001-5,500, 5,501-6,000, 6,001-6,500, 6,501-7,000, 7,001-7,500, 7,501-8,000, 8,001-8,500, 8,501-9,000, 9,001-9,500 or 9,501-9,776 probe molecules.

According to still further features in the described preferred embodiments, the at least two variable region segments are selected from the group consisting of a V-segment, a D-segment and a J-segment.

According to still further features in the described preferred embodiments, the V-segment has a third complementarity determining region specific portion which has an amino acid sequence selected from the group consisting of SEQ ID NOs: 1-23, whereas each probe molecule of the probe set is substantially complementary to at least a portion of the sense or antisense strand of the nucleic acid sequence region of the specific polynucleotide, wherein the portion of the sense or antisense strand encodes the third complementarity determining region specific portion of the V-segment. segment.

According to still further features in the described preferred embodiments, each probe molecule of the probe set is a single stranded polynucleotide composed of a number of nucleotides selected from a range of 24-48 nucleotides.

According to still further features in the described preferred embodiments, the single stranded polynucleotide is a single stranded DNA molecule.

According to still further features in the described preferred embodiments, the single stranded polynucleotide includes at least one nucleic acid sequence selected from the group consisting of SEQ ID NOs: 24-60 and antisense sequences thereof.

According to still further features in the described preferred embodiments, the antigen receptor chain is a T-cell receptor chain.

According to still further features in the described preferred embodiments, the T-cell receptor chain is T-cell receptor beta.

According to still further features in the described preferred embodiments, the antigen receptor chain is a human antigen receptor chain.

The present invention successfully addresses the shortcomings of the presently known configurations by providing an optimal method of typing antigen receptor specificity repertoires.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-b are diagrams depicting the array-level and subarray-level layout, respectively, of the degenerate probes immobilized on the microarray. Subarrays 1-13 of the slide were each printed with the set of oligonucleotide probes capable of specifically hybridizing with the target cDNAs of TCRβ variable regions having the indicated Jβ-segments (FIG. 1a). The actual subarray printing pattern is shown in FIG. 1b. Namely, within each subarray, triplicate cells were printed with the degenerate probe capable of specifically hybridizing with target cDNAs of TCRβ variable regions having the Jβ-segment specific to the subarray and a Vβ-segment belonging to one of the 23 novel Vβ-segment groups, as indicated in each cell. Three cells of each subarray are printed with Cy3 for marking the orientation of the subarray.

FIG. 2 is a fluorescence photomicrograph of a microarray analysis depicting specific high affinity hybridization of cDNA of a specific TCRβ chain to its corresponding subarray and to cells within the subarray corresponding to the novel Vβ-segment group to which its Vβ-segment belongs. The cDNA analyzed was of a TCRβ chain whose variable region includes a Jβ2.1-segment and a Vβ-segment belonging to novel Vβ-segment group No. 4 (having a CDR3 with a Vβ specific portion consisting of a CAS amino acid sequence motif, Table 1). Note hybridization of the cDNA to the Jβ2.1 specific subarray, and to cells within this subarray specific for Vβ4, Vβ16 and Vβ18 which each belong to novel Vβ-segment group No. 4. The hybridized microarray was analyzed for Cy5 fluorescence.

FIG. 3 is a fluorescence photomicrograph of a microarray analysis depicting a highly distinctive pattern of hybridization of target cDNAs representing an individual's TCRβ specificity repertoire to the set of 299 degenerate oligonucleotide probes of the present invention. Three cells of each subarray were printed with Cy3 for marking the orientation of the subarray.

FIG. 4 is a schematic diagram depicting a probe array.

### DESCRIPTION OF THE PREFERRED EMBODIMENTS

The present invention is of methods of typing a variable region of an antigen receptor chain, of probe arrays for performing such typing, and of probe sets for generating such probe arrays. Specifically, the present invention can be used for optimally typing an antigen receptor specificity repertoire in an individual. As such, the methods, probe arrays and probe sets of the present invention can be used for optimally typing an antigen receptor specificity repertoire so as enable optimal identification of a specificity pattern correlated with an immunological phenotype specific to a disease associated with a protective or pathogenic antigen specific immune response. Due to the critical importance of such immunological phenotyping for medical management thereof, the present invention can be used to enable optimal medical management of such diseases.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

No optimal methods are available for medical management (e.g., prevention, diagnosis, treatment, patient monitoring, prognosis, drug design, and the like) of the vast range of lethal/debilitating antigen associated diseases, i.e., diseases associated with a protective or pathogenic antigen specific immune response, such as infectious, autoimmune, transplantation related, malignant, allergic, malignant and inflammatory diseases. An optimal strategy for facilitating medical management of such a disease in an individual would be via a method enabling optimal typing of an antigen receptor specificity repertoire thereof. Such typing could be used to optimally qualify the antigen receptor specificity repertoire of the individual with respect to a reference specificity pattern which is known to correlate with a phenotype associated with the disease. Such qualification could then be used to optimally characterize the phenotype of the individual, and hence could be used to optimize medical management of the disease in the individual. Such a typing method could further be used to enable identification of a novel specificity pattern shared among individuals sharing a phenotype associated with such a disease.

Various methods of typing antigen receptor specificities have been described by the prior art.

Such approaches include those based on typing antigen receptor specificities as a function of length of a CDR, genetic transformation of host cells for analysis of cloned antigen receptor chains, random sequencing of variable region encoding sequences, RNase protection assays, variable region segment typing using monoclonal antibodies (mAb's), PCR amplification of specific variable region segments from cDNA of cell/tissue samples and analysis of products via Southern blotting or single-strand conformation polymorphism (SSCP), or semi quantitative PCR techniques, antigen receptor antigen microarrays, and microarrays utilizing oligonucleotide probes specific for individual variable region segments.

However, all such prior art approaches suffer from various drawbacks, including being suboptimally informative with respect to antigen receptor specificities per se, being impracticably labor intensive/unadaptable to high throughput methodology, and/or being restricted to very limited numbers of antigen receptor specificities due to unavailability of probe reagents. Most importantly, no prior art approach enables optimal typing of an antigen receptor chain specificity repertoire as a function of rearranged variable region segment combinations.

Thus, all prior art approaches have failed to provide adequate solutions for typing antigen receptor specificity repertoires.

While conceiving the present invention it was hypothesized that methods enabling typing of an antigen receptor variable region segment rearrangement repertoire according to specific combinations of at least two variable region segments could be used to optimally type the repertoire of specificities of the antigen receptor in an individual, and that such typing could therefore facilitate optimal medical management of an antigen associated disease.

While reducing the present invention to practice, a unique probe set was designed and synthesized, and used for producing a unique probe array which was used for optimally typing the T-cell receptor beta segment specificity repertoire of a human individual.

Hence, the present method traverses many of the limitations of the prior art.

Thus, according to the present invention, there is provided a method of typing a variable region of a specific variant of an antigen receptor chain. The method is effected in a first step by exposing a probe set to a sense or antisense strand of a polynucleotide encoding at least a portion of the variable region of the specific variant of the antigen receptor chain, the probe set including a plurality of probe molecules each of which being substantially complementary to a sense or antisense strand of a nucleic acid sequence region of a specific polynucleotide encoding a variant of the antigen receptor chain, where the nucleic acid sequence region of the specific polynucleotide distinctly encodes a specific combination of at least two variable region segments of the antigen receptor chain. In a second step, the method is effected by measuring hybridization of each probe molecule of the plurality of probe molecules with the sense or antisense strand of the nucleic acid sequence region of the polynucleotide encoding at least a portion of the variable region of the specific variant of the antigen receptor chain.

By way of illustration, a nucleic acid sequence which “distinctly encodes” a specific combination of at least two variable segments of an antigen receptor chain has a unique nucleic acid sequence encoding at least a portion of each of at least two variable region segments of the antigen receptor chain relative to all possible nucleic acid sequences encoding at least a portion of each of at least two variable region segments of the antigen receptor chain. Thus, examples of nucleic acid sequences which distinctly encode a specific combination of at least two variable region segments of the antigen receptor chain with respect to each other include: (i) nucleic acid sequences which encode, with different nucleic sequences and/or with at least one difference in codon usage, a specific segment of the variable region of the antigen receptor chain (i.e., nucleic acid sequences which encode polypeptides having identical amino acid sequences); and (ii) where the antigen receptor chain is of a type having three types of variable region segments (e.g., V-, D-, and J-segments, as in the case of human T-cell receptor beta), nucleic acid sequences which encode segments of the variable region of the antigen receptor chain having identical amino acid sequences specific to two types of variable region segments of the antigen receptor chain (e.g., the V-segment and the J-segment), but which differ in having non-identical amino acid sequences specific to a third type of variable region segment (e.g., the D-segment).

As used herein, the term “specific combination of at least two variable region segments of the antigen receptor chain” refers to a combination of variable region segments encoded by gene segments belonging to a specific combination of at least two types of variable region genes (i.e., irrespective of the particular variable region gene segments encoding the at least two variable region segments.

Since a probe molecule of the present invention is substantially complementary to a sense or antisense strand of a polynucleotide having nucleic acid sequence region distinctly encoding a specific combination of at least two variable region segments of a variant of the antigen receptor chain, and since the antigenic specificity of a specific variant of an antigen receptor chain is primarily determined by its particular combination of variable region segments, the method of the present invention can be used for optimally typing the repertoire of antigenic specificities of an antigen receptor chain of an individual (referred to hereinafter as “specificity repertoire”). Hence, the method can be used for optimally qualifying such a specificity repertoire with respect to a reference specificity pattern which is known to correlate with a phenotype related to an antigen associated disease, and thereby can be used for optimally qualifying such an individual with respect to such a phenotype. Since such qualification enables optimal performance of numerous aspects of medical management of such a disease in an individual, including prevention, diagnosis, treatment, patient monitoring, prognosis, and drug design, the method of the present invention therefore enables optimal medical management of such a disease in an individual. Furthermore, the method can be used to identify a novel reference specificity pattern by enabling typing of the specificity repertoire in a group of individuals sharing a phenotype related to an antigen associated disease. The resultant set of specificity repertoires may then be analyzed to identify the novel reference specificity pattern common to all or a defined proportion of which.

As used herein, the phrase “antigen associated disease” refers to any disease associated with a protective antigen specific immune response, potentially associated with a protective antigen specific immune response, or associated with a pathogenic antigen specific immune response.

As used herein, the term “disease” refers to any medical disease, disorder, condition, or syndrome, or to any undesired and/or abnormal physiological morphological, and/or physical state and/or condition.

The method of the present invention may be effected in any of various ways, depending on the application and purpose, including via use of any of various types of samples of analyte strands, via use of a probe set which includes any of various probe molecule pluralities, and via performing the exposure and hybridization measurement steps of the method in any of various ways.

As used herein, the phrase “analyte strand” refers to a sense or antisense strand of a polynucleotide encoding at least a portion of the variable region of a specific variant of the antigen receptor chain.

Thus, the present invention provides a probe set which comprises a plurality of probe molecules, each of which being substantially complementary to a sense or antisense strand of a nucleic acid sequence region of a specific polynucleotide encoding a variant of an antigen receptor chain, where the nucleic acid sequence region distinctly encodes a specific combination of at least two variable region segments of the antigen receptor chain.

The probe set of the present invention may comprise any of various pluralities of probe molecules, depending on the application and purpose. More particularly, the probe set may include any of various numbers of distinct probe molecules of the present invention, distinct probe molecules of the present invention in any of various proportions with respect to each other, a probe molecule of the present invention substantially complementary to any of various analyte strands, a probe molecule of the present invention capable of specifically hybridizing with a target analyte strand with any of various affinities, and/or a probe molecule of the present invention of any of various types.

As used herein, the phrase “target analyte strand” when relating to a specific probe molecule of the present invention refers to an analyte strand of the present invention to which such a probe molecule has been selected to be substantially complementary under specific conditions.

As used herein, a probe molecule of the present invention which is capable of “specifically hybridizing” to a target analyte strand thereof refers to probe molecule having an optimal or unique capacity to substantially hybridize with the target analyte strand under defined conditions relative to the other probe molecules of the probe set.

Preferably, the probe set includes distinct probe molecules selected optimally suitable for typing an analyte strand of the present invention, or a plurality of distinct analyte strands of the present invention, depending on the type of the analyte strand or of the plurality of distinct analyte strands. One of ordinary skill in the art will possess the necessary expertise for selecting a probe set of the present invention which includes distinct probe molecules optimally suitable for typing a particular type of analyte strand of the present invention, or plurality of distinct analyte strands of the present invention.

Preferably, according to the teachings of the present invention, the probe set includes distinct subsets of probe molecules such that each distinct probe molecule of each distinct probe molecule subset is substantially complementary to a sense or antisense strand of a specific polynucleotide encoding a specific combination of at least two variable region segments of the antigen receptor chain where each of the least two variable region segments is encoded by a specific variable region gene.

A probe set of the present invention which includes such distinct probe molecule subsets is optimal for typing a plurality of distinct antigen receptor chains relative to all prior art probe sets since it can be used according to the teachings of the present invention for optimally typing such a plurality of distinct antigen receptor chains according to any given combination of at least two variable region segments.

For enabling optimally flexible and informative typing of a specificity repertoire of an individual, the probe set preferably includes probe molecule subsets selected so as to enable typing the antigen receptor chain specificity repertoire according to a maximal number of different combinations of the at least two variable region segments of the antigen receptor chain.

The probe set may include any of various numbers of distinct subsets of probe molecule of the present invention, depending on the application and purpose. Preferably, the probe set includes a number of distinct subsets of probe molecules selected from a range of 1-299 distinct subsets of probe molecules. Preferably, the probe set includes about 299 distinct subsets of probe molecules, most preferably 299 distinct subsets of probe molecules.

Each distinct subset of probe molecules of the present invention may include any of various numbers of distinct probe molecules. Preferably, each distinct subset of probe molecules includes a number of distinct probe molecules selected from a range of 1-128 distinct probe molecules, more preferably from a range of 16-128 distinct probe molecules.

Depending on the application and purpose, a distinct subset of probe molecules of the present invention may include distinct probe molecules in any of various proportions. The ordinarily skilled artisan will possess the necessary expertise to modify such proportion in order to modulate the specificity bias of a distinct subset of probe molecules of the present invention so as to enable optimally informative typing of the antigen receptor chain.

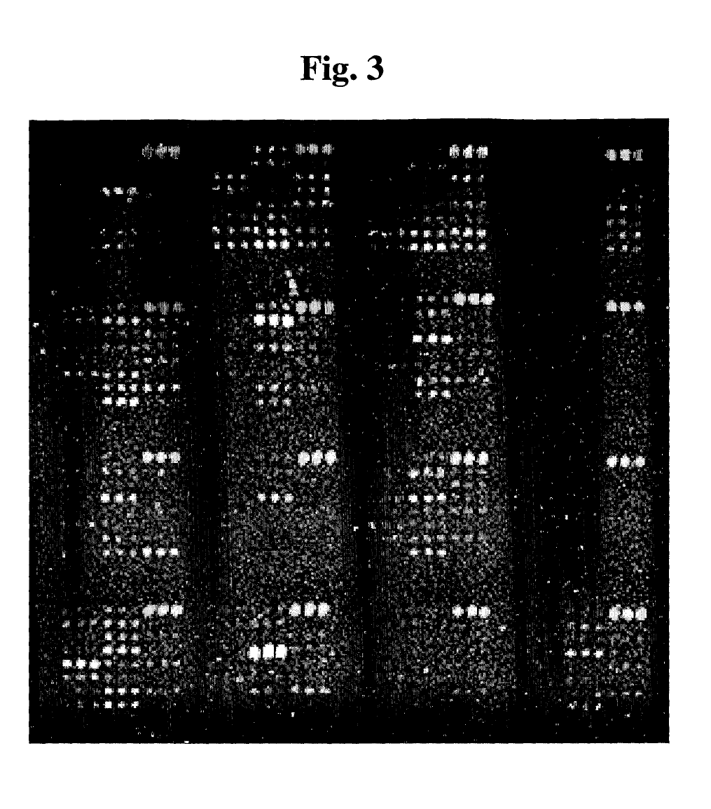

As is described in the Examples section which follows (refer to FIG. 3), a probe set of the present invention including 299 distinct subsets of probe molecules each of which including 16-128 distinct probe molecules can be used for optimally typing a human T-cell receptor beta chain repertoire according to the teachings of the present invention.

Depending on the application and purpose, a probe molecule of the present invention may be selected of any of various types. In particular, a probe molecule of the present invention may be selected having any of various chemical compositions, physical dimensions, and/or molecular weights.

Preferably, a probe molecule of the present invention is a single stranded polynucleotide, most preferably a single stranded DNA molecule, composed of a number of nucleotides selected from a range of 24-48 nucleotides.

Alternately, a probe molecule of the present invention may be a double stranded polynucleotide, or a polypeptide, such as, for example, a peptide or an antibody.

A polynucleotide probe molecule or analyte strand of the present invention may include any combination of any of various different types of nucleotide bases. A polynucleotide probe molecule or analyte strand of the present invention which includes any combination of any of various different types of nucleotide bases may be obtained according to techniques which are well known in the art. Suitable nucleotide bases for preparing a polynucleotide probe molecule or analyte strand of the present invention may be selected from naturally occurring nucleotide bases such as adenine, cytosine, guanine, uracil, and thymine; and non-naturally occurring or non natural/synthetic nucleotide bases such as 8-oxo-guanine, 6-mercaptoguanine, 4-acetylcytidine, 5-(carboxyhydroxyethyl)uridine, 2′-O-methylcytidine, 5-carboxymethylamino-methyl-2-thioridine, 5-carboxymethylaminomethyluridine, dihydro-uridine, 2′-O-methylpseudouridine, β,D-galactosylqueosine, 2′-O-methylguanosine, inosine, N6-isopentenyladenosine, 1-methyladenosine, 1-methylpseeudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, β,D-mannosylqueosine, 5-methoxycarbonylmethyluridine, 5-methoxy-uridine, 2-methylthio-N6-isopentenyladenosine, N-((9-β-D-ribofuranosyl-2-methyl-thiopurine-6-yl)carbamoyl)threonine, N-((9-β-D-ribofuranosylpurine-6-yl)N-methyl-carbamoyl)threonine, unidine-5-oxyacetic acid methylester, uridine-5-oxyacetic acid, wybutoxosine, pseudouridine, queosine, 2-thiocytidine, 5-methyl-2-thiouridine, 2-thiouridine, 2-thiouridine, 5-methyluridine, N-((9-β-D-ribofuranosylpurine-6-yl)carbamoyl)threonine, 2′-O-methyl-5-methyluridine, 2′-O-methyluridine, wybutosine, and 3-(3-amino-3-carboxypropyl)uridine. Any nucleotide backbone may be employed, including DNA, RNA (although RNA is less preferred than DNA), modified sugars such as carbocycles, and sugars containing 2′ substitutions such as fluoro and methoxy. Any of the internucleotide bridging phosphate residues of a polynucleotide probe molecule of the present invention may be modified phosphates, such as methyl phosphonates, methyl phosphonothioates, phosphoromorpholidates, phosphoropiperazidates and phosphoramidates (for example, every other one of the internucleotide bridging phosphate residues may be modified as described). A probe molecule of the present invention may be a “peptide nucleic acid” such as described in P. Nielsen et al., Science 254, 1497-1500 (1991).

A probe molecule of the present invention may be selected substantially complementary to a sense or antisense strand of a specific polynucleotide which encodes a specific combination of at least two variable region segments of the antigen receptor chain via discontinuous nucleic acid sequences, or most preferably, via a continuous nucleic acid sequence thereof. One of ordinary skill in the art will possess the necessary expertise for selecting a probe set of the present invention which includes probe molecules substantially complementary to a sense or antisense strand of a specific polynucleotide which encodes a specific combination of at least two variable region segments of the antigen receptor chain via a discontinuous nucleic acid sequence.

When aligned for maximum complementarity, the maximum percentage of mismatched nucleotide bases between the nucleic acid sequence of a single stranded polynucleotide probe molecule of the present invention and that of a target analyte strand thereof, is preferably 17%, more preferably 16%, more preferably 15%, more preferably 14%, more preferably 13%, more preferably 12%, more preferably 11%, more preferably 10%, more preferably 9%, more preferably 8%, more preferably 7%, more preferably 6%, more preferably 5%, more preferably 4%, more preferably 3%, more preferably 2%, and more preferably 1%. Most preferably, there are no mismatched bases between the nucleic acid sequence of a single stranded polynucleotide probe molecule of the present invention and that of a target analyte strand thereof, such that their nucleotide sequences are fully complementary. The ordinarily skilled artisan will be knowledgeable regarding complementarity relationships between nucleotide base pairs, and will recognize that, where a probe molecule of the present invention is a single stranded polynucleotide, relaxing the stringency of the hybridizing conditions will allow sequence mismatches between the probe molecule and a target analyte strand to be tolerated, and further, that the degree of mismatch tolerated can be controlled by suitable adjustment of the hybridization conditions.

As described hereinabove, a probe molecule of the present invention may be selected capable of specifically hybridizing with a target analyte strand thereof with any of various affinities, depending on the application and purpose.

A probe molecule of the present invention may be advantageously selected capable of specifically hybridizing with a target analyte strand with maximal affinity so as to enable optimally stable hybridization therewith under defined conditions, and thereby optimal detection of hybridization of the probe molecule with a target analyte strand thereof.

A probe molecule of the present invention may be advantageously selected capable of specifically hybridizing, under defined conditions, with a target analyte strand thereof with the same, or about the same affinity, as the specific hybridization of one or more of the other probe molecules of the probe set with their target analyte strands. It will be appreciated that such affinity matching can be used for quantitatively comparing the representation of distinct analyte strands in a mixture of distinct analyte strands of the present invention.

As described hereinabove, depending on the application and purpose, a probe molecule of the present invention may be selected substantially complementary to any of various types of analyte strands of the present invention. In particular, a probe molecule of the present invention may be selected substantially complementary to a sense or antisense strand of a specific polynucleotide which encodes any of various specific combinations of at least two variable region segments of the antigen receptor chain.

Preferably, a probe molecule of the present invention is substantially complementary to a sense strand, more preferably to an antisense strand, of a polynucleotide which encodes a specific combination of a V-segment, a D-segment and a J-segment. It will be appreciated that in the case of human antigen receptor chains the variable region of an immunoglobulin heavy chain, T-cell receptor beta chain, or T-cell receptor delta chain may include a D-segment, whereas that of an immunoglobulin light chain, T-cell receptor alpha chain, or T-cell receptor gamma chain will generally not include a D-segment.

Preferably, a probe molecule of the present invention is substantially complementary to a sense or antisense strand of a specific polynucleotide having a nucleic acid sequence region which distinctly encodes at least a portion of the amino acid sequence, more preferably at least the entire, amino acid sequence of, a complementarity determining region (CDR), preferably a third complementarity determining region (CDR3), of the antigen receptor chain.

Ample information regarding nucleotide sequences, amino acid sequences, variable region segment alleles, variable region genes, variable region gene segments, genetic polymorphisms, CDRs, and the like, relating to antigen receptor chains is readily available (refer, for example to: http://imgt.cines.fr), and can be used by one of ordinarily skill to practice the various embodiments of the method of the present invention according to the teachings of the present invention.

As is described and illustrated in the Examples section which follows, while conceiving the present invention, the present inventors devised a novel classification method whereby essentially any expressed human T-cell receptor beta chain variable region V-segment can be classified as belonging to one of 23 novel human T-cell receptor Vbeta-segment groups according to which one of the amino acid sequences set forth in SEQ ID NOs: 1-23 corresponds to the third complementarity determining region (CDR3) specific portion of the V-segment. Use of this novel classification scheme for typing of a human T-cell receptor beta chain specificity repertoire is a particularly advantageous feature of the present invention since it obviates the excessively cumbersome and complex prior art requirement of typing human T-cell receptor beta chain V-segments according to each one of the approximately 128 distinct gene sequences encoding such expressed variable region segments identified to date.

Preferably, a single stranded polynucleotide probe molecule of the present invention includes at least one nucleic acid sequence selected from SEQ ID NOs: 24-60 or, less preferably antisense sequences thereof. More preferably, a single stranded polynucleotide probe molecule of the present invention includes: (i) a nucleic acid sequence selected from SEQ ID NOs: 24-46 or, less preferably, antisense sequences thereof; (ii) a nucleic acid sequence selected from the set of nucleic acid sequences of SEQ ID NO: 47 or, less preferably antisense sequences thereof; and/or (iii) a nucleic acid sequence selected from SEQ ID NOs: 48-60 or, less preferably, antisense sequences thereof. More preferably, a single stranded polynucleotide probe molecule of the present invention includes: (i) a nucleic acid sequence selected from SEQ ID NOs: 24-46 or, less preferably, antisense sequences thereof; (ii) a nucleic acid sequence selected from the set of nucleic acid sequences of SEQ ID NO: 47 or, less preferably, antisense sequences thereof; and (iii) a nucleic acid sequence selected from SEQ ID NOs: 48-60 or, less preferably, antisense thereof. More preferably a single stranded polynucleotide probe molecule of the present invention comprises a nucleic acid sequence which includes, in one contiguous sequence from 5′ to 3′, a nucleic acid sequence selected from SEQ ID NOs: 24-46, a nucleic acid sequence selected from the set of nucleic acid sequences of SEQ ID NO: 47, and a nucleic acid sequence selected from SEQ ID NOs: 48-60.

As is described and illustrated in the Examples section which follows (refer to Table 1 in particular), the nucleic acid sequences of SEQ ID NOs: 24-46 are complementary to antisense sequences of polynucleotides encoding carboxy terminal portions of human T-cell receptor Vbeta-segments belonging to specific groups of the above described novel human T-cell receptor Vbeta-segment groups, where such carboxy terminal portions essentially consist of, or essentially include, CDR3 specific portions of such variable region segments. As such, single stranded polynucleotide probe molecules of the present invention which include a sequence selected from SEQ ID NOs: 24-46, or antisense sequences thereof, can be used, according to the teachings of the present invention, for typing human T-cell receptor beta chains according to a specific combination of at least two variable region segments including a V-segment thereof, where such V-segment is typed according to the novel human T-cell receptor Vbeta-segment group classification of the present invention. As is also described and illustrated in the Examples section which follows (refer to Table 2 in particular), the nucleic acid sequences of SEQ ID NOs: 48-60 are complementary to antisense sequences of polynucleotide sequences encoding amino terminal portions of human T-cell receptor beta J-segments where such amino terminal portions essentially consist of, or essentially include, CDR3 specific portions of such variable region segments. As such, single stranded polynucleotide probe molecules of the present invention which include a sequence selected from SEQ ID NOs: 48-60, or antisense sequences thereof, can be used, according to the teachings of the present invention, for typing human T-cell receptor beta chains according to a specific combination of at least two variable region segments including a J-segment thereof. As is further described and illustrated in the Examples section which follows, the nucleic acid sequences of SEQ ID NO: 47 are complementary to antisense sequences of polynucleotides encoding human T-cell receptor beta D-segments. As such, single stranded polynucleotide molecules of the present invention which include a sequence selected from the nucleic acid sequences of SEQ ID NO: 47, or antisense sequences thereof, can be used for typing human T-cell receptor beta chains according to a specific combination of variable region segments including a D-segment thereof.

As is described and illustrated in the Examples section which follows (refer to FIG. 3), a probe set of the present invention which comprises a plurality of probe molecules each of which including in one contiguous sequence from 5′ to 3′, a nucleic acid sequence selected from SEQ ID NOs: 24-46, a nucleic acid sequence selected from the set of nucleic acid sequences of SEQ ID NO: 47, and a nucleic acid sequence selected from SEQ ID NOs: 48-60 can be used, according to the teachings of the present invention, for optimally typing a human T-cell receptor beta chain specificity repertoire (refer to FIG. 3). Table 3 of the Examples section below lists representative examples of single stranded DNA probe molecules of the present invention (SEQ ID NOs: 61-73) which include in one contiguous sequence from 5′ to 3′, the nucleic acid sequence of SEQ ID NO: 24, a nucleic acid sequence selected from the set of nucleic acid sequences of SEQ ID NO: 47, and a nucleic acid sequence selected from SEQ ID NOs: 48-60. By way of example, the single stranded DNA probe molecules described in Table 3 can be used, according to the teachings of the present invention, for typing human T-cell receptor beta chains according to a specific combination of variable region segments which includes: (i) a V-segment with CDR3 specific amino acids essentially consisting of cysteine residue at the carboxy terminus of the V-segment (i.e., belonging to novel Vbeta segment group No. 1 (refer to Table 1 of the Examples section below); and (ii) one of thirteen Jbeta segments.

As described hereinabove, the probe set may include any of various numbers of distinct probe molecules, depending on the application and purpose.

Preferably, the probe set includes a number of probe molecules selected from 2, 3, 4, 5, 6, 7, 8, 9, 10, 11, 12, 13, 14, 15, 16, 17, 18, 19, 20, 21, 22, 23, 24 or 25 distinct probe molecules of the present invention, or selected from a range of 26-30, 31-35, 36-40, 41-45, 46-50, 51-55, 56-60, 61-65, 66-70, 71-75, 76-80, 81-85, 86-90, 91-95, 96-100, 101-150, 151-200, 201-250, 251-300, 301-350, 351-400, 401-450, 451-500, 501-550, 551-600, 601-650, 651-700, 701-750, 751-800, 801-850, 851-900, 901-950, 951-1,000, 1,001-1,100, 1,101-1,200, 1,201-1,300, 1,301-1,400, 1,401-1,500, 1,501-1,600, 1,601-1,700, 1,701-1,800, 1,801-1,900, 1,901-2,000, 2,001-2,100, 2,101-2,200, 2,201-2,300, 2,301-2,400, 2,401-2,500, 2,501-2,600, 2,601-2,700, 2,701-2,800, 2,801-2,900, 2901-3000, 3,001-3,500, 3,501-4,000, 4,001-4,500, 4,501-5,000, 5,001-5,500, 5,501-6,000, 6,001-6,500, 6,501-7,000, 7,001-7,500, 7,501-8,000, 8,001-8,500, 8,501-9,000, 9,001-9,500 or 9,501-9,776 probe molecules of the present invention. More preferably, the probe set includes about 9,776 distinct probe molecules of the present invention. Most preferably, the probe set includes 9,776 distinct probe molecules of the present invention.

As used herein the term “about” refers to plus or minus 10%.

As is described and illustrated in the Examples section which follows, a probe set of the present invention which includes 9,776 distinct probe molecules can be used for optimally typing a human T-cell receptor beta chain specificity repertoire.

As described hereinabove, exposing the probe set to the analyte strand and measuring hybridization of the analyte strand with probe molecules of the present invention may be effected in any of various ways, depending on the application and purpose.

Preferably, the probe set is exposed to the analyte strand sample in such a way as to enable specific hybridization of probe molecules of the probe set with target analyte strands thereof present in the analyte strand sample, and to enable subsequent measurement of such hybridization.

When using the method of the present invention for qualifying a specificity repertoire of the present invention with respect to a reference specificity pattern of the present invention, the exposure step is preferably effected in such a way as to optimally enable such qualification. When using the method of the present invention for identifying a reference specificity pattern of the present invention, the exposure step is preferably effected in such a way as to optimally enable such identification. It will be appreciated that to optimally enable such specificity pattern identification or specificity repertoire qualification, the exposure step may be advantageously effected in such a way as to preferentially enable hybridization of specific analyte strands of the analyte strand sample. Such preferential hybridization may be achieved by the skilled artisan by selecting suitable hybridization conditions during the exposure of the probe set with the analyte strand sample.

The probe set can be exposed to the analyte strand sample in solution by forming a set of solutions each of which containing one or more analyte strands of the analyte strand sample and one or more distinct probe molecules of the probe set, such that each distinct probe molecule of the probe set is contained in at least one, more preferably only one, solution of the set of solutions. Preferably, each solution of such a solution set is formed under hybridization conditions suitable for enabling specific hybridization of probe molecules with target analyte strands thereof contained therein.

It will be well within the purview of one of ordinary skill to select such suitable hybridization conditions. Ample guidance for selecting such hybridization conditions is provided in the literature of the art (refer, for example to: U.S. Pat. No. 6,551,784; U.S. Pat. No. 4,358,535 to Falkow et al. and other U.S. Patent references citing the same).

Any of various methods known to one of ordinary skill in the art may be employed for measuring the hybridization of probe molecules of the present invention with target analyte strands thereof in a solution. Such methods include, for example, fluorescence resonance energy transfer (FRET) based methods. Ample guidance for practicing such FRET based methods is available in the literature of the art (see, for example: Gakamsky D. et al., “Evaluating Receptor Stoichiometry by Fluorescence Resonance Energy Transfer,” in “Receptors: A Practical Approach,” 2nd ed., Stanford C. and Horton R., eds., Oxford University Press, UK. (2001); for high throughput applications of FRET refer, for example, to: Stenroos K. and Hurskainen P. 1998. Cytokine 495:5; and Kane S A. et al., 2000. Anal. Biochem. 278:29).

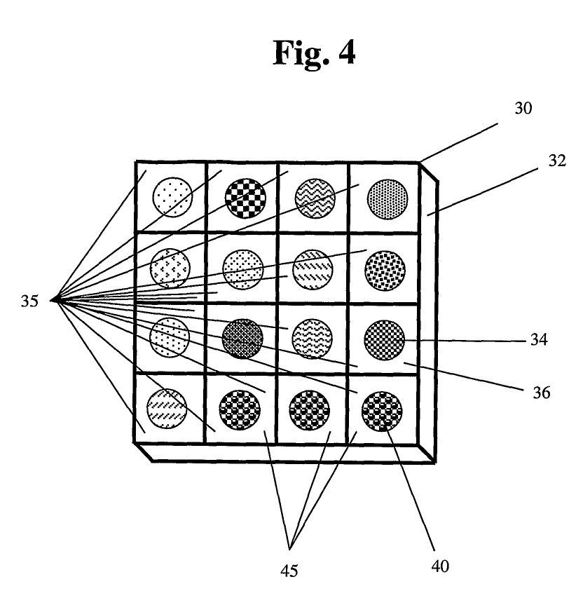

Preferably, the probe set of the present invention is bound to a support so as to form a probe array, such as a microarray. An example of a probe array is illustrated in FIG. 4. Probe array 30 includes a support 32 which can be fabricated from glass and shaped so as to form an upward-facing planar surface. Support 32 includes a plurality of addressable locations (each indicated by 35) which can be configured as wells, microwells, or areas delineated by grid etchings. Each probe molecule or distinct subset of probe molecules 34 of the probe set of the present invention is immobilized to a specific addressable location 36 of probe array 30. Such immobilization can be effected via covalent or non-covalent interactions between the probe molecules and the surface of the array support or between support bound linker molecules and the probe molecules. A detectable label 40 is immobilized to each of a set of reference addressable locations (each indicated by 45) so as to provide a reference point for identifying each addressable location (indicated by 35).

Various types of probe arrays may be used, depending on the application and purpose. Suitable types of probe arrays for practicing the method of the present invention may be referred to in the art variously as DNA or oligonucleotide microarrays, DNA or oligonucleotide chips, or DNA or oligonucleotide biochips. Large numbers of distinct analyte strands of the present invention, may be analyzed simultaneously using a probe array of the present invention, allowing precise high throughput measurement of the hybridization of immobilized probe molecules of the present invention with target analyte strands thereof.

Various methods have been developed for preparing probe arrays. State-of-the-art methods involves using a robotic apparatus to apply or “spot” distinct solutions containing probe molecules to closely spaced specific addressable locations on the surface of a planar support, typically a glass support, such as a microscope slide, which is subsequently processed by suitable thermal and/or chemical treatment to attach probe molecules to the surface of the support. Suitable supports may also include silicon, nitrocellulose, paper, cellulosic supports, and the like.

Ample guidance for obtaining and utilizing a probe array for suitably practicing the method of the present invention is provided in the literature of the art (for example, refer to: U.S. Pat. No. 6,551,784; U.S. Pat. No. 6,251,601; Forster et al., 2003. J Endocrinol. 178:195-204; Howbrook et al., 2003. Drug Discov Today 8:642-51; Xiang et al., 2003. Curr Opin Drug Discov Devel. 6:384; Hardiman G., 2003. Pharmacogenomics 4:251). Custom designed arrays can be purchased from commercial suppliers [for example, Affymetrix, Santa Clara, USA; or Agilent Technologies, Palo Alto, USA).

The probe array may include the plurality of addressable locations at any of various surface densities, depending on the application and purpose.

A probe array of the present invention which includes specific addressable locations at a surface density of at least 625 specific addressable locations per square centimeter thereof may be advantageously used to practice the method of the present invention.

By virtue of being practicable using a probe array having a support including the plurality of addressable locations at a surface density of at least 625 specific addressable locations per square centimeter, the method of the present invention may be practiced at high throughput rates and volumes, and as such is advantageous over prior art methods of typing the variable region of the specific variant of the antigen receptor chain.

In addition to probe molecules of the probe set, the array may advantageously include control probe molecules. Such control probe molecules may include normalization control probes, and/or expression level control probes.

Normalization control probes are probe molecules that are perfectly complementary to labeled reference oligonucleotides that are included in the hybridization solution. The signals obtained from the normalization control probes after hybridization provide a control for variations in hybridization conditions, label intensity, “reading” efficiency and other factors that may cause the signal of a perfect hybridization to vary between arrays. For example, signals, such as fluorescence intensity, read from all other probe molecules of the probe array are divided by the signal (e.g., fluorescence intensity) from the normalization control probes thereby normalizing the measurements.

Since hybridization efficiency varies with base composition and probe length, polynucleotide normalization control probes may be selected to reflect the average length of single stranded polynucleotide probe molecules of the present invention, or multiple normalization control probes may be selected to cover a range of lengths of single stranded polynucleotide probe molecules of the present invention. Normalization control probes may be selected to reflect the base composition of the probe molecules of the probe set. Preferably, normalization control probes are incapable of substantially hybridizing with an analyte strand of the analyte strand sample. Normalization control probes can be bound to various addressable locations the probe array to control for spatial variation in hybridization efficiently. Preferably, normalization control probes are located at the corners or edges of the array to control for edge effects, as well as in the middle of the array.

Expression level control probes are probe molecules that hybridize specifically with polynucleotides derived from housekeeping gene mRNA expressed in the cells from which mRNA was used to derive an analyte strand sample of the present invention, and may therefore be used to provide a normalization reference for comparing expression levels of different variants of the antigen receptor chain. Suitable housekeeping genes include the genes for beta-actin, transferrin receptor, GAPDH, and the like.

Any of various numbers of distinct probe molecules of the present invention or distinct subsets of probe molecules of the present invention may be attached to a specific addressable location of the probe array, depending on the application and purpose.

Preferably, each probe molecule, or distinct subset of probe molecules of the present invention, which is attached to a specific addressable location of the array is attached independently to at least two, more preferably to at least three separate specific addressable locations of the array in order to enable generation of statistically robust data when performing the hybridization measurement step of the method.

Preferably, exposing an array-immobilized probe set of the present invention to the analyte strand sample is effected according to the protocol set forth in the Examples section below.

As described hereinabove, the method may be effected using any of various types of samples of analyte strand, depending on the application and purpose.

Exposing the probe set to the analyte strand may be effected by exposing the probe set to a sample of analyte strands of the present invention (referred to hereinafter as “analyte strand sample”) which may be composed of any of various homogeneous or heterogeneous populations of distinct analyte strands of the present invention.

It will be appreciated that for typing the specificity of a plurality of distinct variable region variants of an antigen receptor, such as a specificity repertoire of the present invention, the analyte strand sample will preferably include a number of distinct analyte strands suitably representing the repertoire.

The method of the present invention may be practiced using any of various types of analyte strand.

Preferably, the analyte strand is an antisense strand of the analyte polynucleotide, more preferably a complementary DNA (cDNA) strand of the polynucleotide.

As is described and illustrated in the Examples section which follows, the method of the present invention may be successfully practiced using an analyte strand which is a cDNA molecule.

The analyte strand may be a sense or antisense strand of a polynucleotide encoding any of various portions of the variable region of an antigen receptor chain which may be of any of various types and/or which may be derived from a vertebrate organism of any of various species.

The antigen receptor chain is preferably a T-cell receptor chain, more preferably a T-cell receptor beta chain.

Alternately, the antigen receptor chain may be a T-cell receptor alpha chain, a T-cell receptor gamma chain, a T-cell receptor delta chain, an immunoglobulin heavy chain (e.g., a gamma, mu, alpha, delta or epsilon isotype heavy chain), or an immunoglobulin light chain (e.g., kappa or lambda light chain).

Preferably, the vertebrate organism is a mammal, most preferably a human.

As is illustrated and described in the Examples section which follows, the method of the present invention may be effectively practiced where the antigen receptor chain is human T-cell receptor beta chain.

The analyte strand may be obtained from any of various cell types, depending on the application and purpose.

In the case where the antigen receptor chain is a T-cell receptor chain, the cells will generally be T-lymphocytes, and in the case where the antigen receptor chain is an immunoglobulin chain, the cells will preferably be B-lymphocytes, such cell types normally expressing such respective antigen receptor chain types. Alternately, the cells may be of any type which includes a polynucleotide encoding at least a portion of the variable region of the antigen receptor chain as a result of genetic transformation.

The cells are preferably primary cells derived from the organism. Alternately, the cells may be derived from cultured cell lines.

The cells may be derived from any of various body parts/fluids of the organism, depending on the application and purpose. Preferably, the cells are derived from peripheral blood of the organism, more preferably from peripheral blood mononuclear cells (PBMCs).

It will be appreciated that peripheral blood is normally the most convenient, safe and non-invasive source from which to obtain significant numbers of lymphocytes from an organism. Peripheral blood mononuclear cells (PBMCs) may be conveniently isolated from peripheral blood using standard density gradient centrifugation methods, such as discontinuous density gradient centrifugation over a Ficoll cushion. Peripheral blood mononuclear cells of a desired type may also be isolated from blood via leukopheresis.

Alternatively, the cells may be derived from a body fluid of the organism such as synovial fluid, cerebrospinal fluid, lymph, bronchioalveolar lavage fluid, gastrointestinal secretions, saliva, urine, feces, or lacrymal secretion. The cells may be derived from any of various tissues of the organism, for example, from a tissue biopsy. When typing a specificity repertoire so as to identify a specificity pattern relating to a particular disease, the selection of an appropriate cell source will be apparent to those of ordinary skill. In particular, when typing a specificity repertoire of the present invention so as to identify a specificity pattern relating to a disease affecting a specific body part/fluid of the organism, the cells may be advantageously derived from such a body part/fluid, of the organism. For example, to identify a specificity pattern relating to an autoimmune disorder affecting the joints (for example, rheumatoid arthritis), synovial fluid of the organism will be the preferred source for the cells, or to identify a specificity pattern relating to a hepatic disease (for example, hepatitis, or primary biliary cirrhosis), liver tissue is an advantageous tissue from which to derive the cells.

The analyte strand may be derived directly from a mixed population of cells, such as non-fractionated PBMCs, or it may be derived from isolated B-lymphocytes or T-lymphocytes. Lymphocytes displaying any desired surface marker may be effectively isolated from a cell suspension, such as a PBMC suspension, by various common art techniques, such as fluorescence activated cell sorting (FACS), magnetic cell sorting (MACS), inter alia.

Preferably, in order to type a specificity repertoire of the present invention, a sufficient number of cells are obtained from the organism so as to derive therefrom a an analyte strand sample of the present invention suitably representing such a repertoire. One ordinarily versed in the art will possess the required expertise for determining a suitable number of cells from which to derive such an analyte strand sample. Where the antigen receptor chain is human T-cell receptor beta chain, a suitable number of peripheral blood mononuclear cells (PBMCs) which may be harvested from an individual to obtain an analyte strand sample adequately representing the repertoire of this chain in the individual is about 50 million cells to 500 million cells, more preferably about 100 million cells.

As is described and illustrated in the Examples section below, T-cell receptor beta specificity repertoire of a human individual may be conveniently typed according to the teachings of the present invention using 100 million PBMCs harvested from the individual.

In cases where the number of the cells of the present invention which may be obtained is restricted to suboptimally low numbers, any of various methods commonly practiced by the ordinarily skilled artisan may be employed for in-vitro expansion of such limited numbers of such cells. In the case of T-lymphocytes, such methods include, for example, stimulation with immobilized or cross-linked anti-CD3 antibodies (optionally in conjunction with stimulation with anti-CD28 antibodies), phytohemagglutinin (PHA), concanavalin (ConA), or pokeweed mitogen (PWM), any of which optionally followed by IL-2 stimulation. Where the cells of the present invention are B-lymphocytes, such methods include, for example, stimulation with pokeweed mitogen (PWM) or bacterial lipopolysaccharide (LPS).

The analyte strand may be derived from cellular polynucleotides using any of various methods commonly practiced in the art, depending on the application and purpose.

A cDNA analyte strand of the present invention may be conveniently derived from cells by isolation of total mRNA thereof, and by using the mRNA as a template for reverse transcription of the cDNA analyte strand. Preferably, reverse transcription is effected using a primer or primers suitable for reverse transcribing a particular cDNA analyte strand. For example, a suitable primer for reverse transcribing from human mRNA a human T-cell receptor beta chain cDNA analyte strand is set forth under SEQ ID NO: 74. Suitable primers for generating a cDNA analyte strand representing any of various specific types or subsets of antigen receptor chain will be known to the skilled artisan (for example, refer to: Kiippers et al., 1993. EMBO J. 12:4955-67; Roers et al., 2000. Am J Pathol. 156:1067-71; Willenbrock et al., 2001. Am J Pathol. 158:1851-7; Muschen et al., 2001. Lab Invest. 81:289-95).

Methods of isolating total mRNA from cells, such as those of the present invention, are well known to those of skill in the art [refer, for example, to: Chapter 3 of Laboratory Techniques in Biochemistry and Molecular Biology: Hybridization With Nucleic Acid Probes, Part I, Theory and Nucleic Acid Preparation, P. Tijssen, ed. Elsevier, N.Y. (1993); and Chapter 3 of Laboratory Techniques in Biochemistry and Molecular Biology: Hybridization With Nucleic Acid Probes, Part I, Theory and Nucleic Acid Preparation, P. Tijssen, ed. Elsevier, N.Y. (1993)].

Non specific mRNA may be eliminated from an RNA sample according to various commonly practiced techniques so as to decrease background signal and improve sensitivity of the hybridization measurement (refer, for example, to: for example, refer to: U.S. Pat. No. 6,551,784).

Preferably, a cDNA analyte strand of the present invention is obtained as described in the Examples section which follows.

Optionally, an analyte strand of the present invention may be obtained by polymerase chain reaction (PCR) amplification from a polynucleotide using suitable primers, as described hereinbelow, and as described in the Examples section which follows. It will be appreciated that PCR amplification may be advantageously employed in order to amplify and/or modify the analyte strand, depending on the application and purpose. One of ordinary skill in the art will possess the necessary expertise for performing PCR amplification of an analyte strand of the present invention from a polynucleotide. Suitable primers for amplifying a sense or antisense strand of a polynucleotide encoding a desired type of antigen receptor or portion thereof and guidance for their use will be known to the skilled artisan (refer, for example, to: Kiippers et al., 1993. EMBO J. 12:4955-67; Roers et al., 2000. Am J Pathol. 156:1067-71; Willenbrock et al., 2001. Am J Pathol. 158:1851-7; Muschen et al., 2001. Lab Invest. 81:289-95). In order to amplify an analyte strand of the present invention from a polynucleotide encoding a human immunoglobulin refer, for example, to: Sblattero and Bradbury, 1998. Immunotechnology 3:271-8; and Wang and Stollar, 2000. J Immunol Methods. 244:217-25.

One of skill in the art will appreciate that when performing PCR amplification of a sample of heterogeneous analyte strands of the present invention, care must be taken to use a method that maintains or controls for the relative frequencies of distinct analyte strands in such a sample to achieve quantitative amplification of such strands.

Methods of “quantitative” amplification are well known to those of skill in the art. For example, quantitative PCR involves simultaneously co-amplifying known concentrations of a control analyte strand using the same primers so as to provide a set of internal standards that may be used to calibrate the PCR reaction. Detailed protocols for quantitative PCR are provided in the literature of the art, for example, refer to “PCR Protocols, A Guide to Methods and Applications”, Innis et al., Academic Press, Inc. New York, (1990).

As is described and illustrated in the Examples section which follows, the method of the present invention may be successfully practiced where the analyte strand is a PCR amplified cDNA molecule.

Preferably, the analyte strand is conjugated with a detectable label so as to enable measurement of hybridization thereof with a probe molecule of the present invention. The analyte strand may be conjugated with any of various types of detectable labels, depending on the application and purpose, via any of various suitable methods known to one of ordinary skill in the art.

While the analyte strand may be conjugated with any of various types of detectable labels, the label is preferably a fluorophore.

Preferably, the fluorophore is Cy5. Alternately, the fluorophore may be any of various fluorophores, including Cy3, fluorescein isothiocyanate (FITC), phycoerythrin (PE), rhodamine, Texas red, and the like. As is described and illustrated in the Examples section below, the method may be performed using Cy5 as the fluorophore. Ample general guidance regarding fluorophore selection, and methods of conjugating a fluorophore to a polynucleotide such as the analyte strand, is available in the literature of the art [refer, for example, to: Richard P. Haugland, “Molecular Probes: Handbook of Fluorescent Probes and Research Chemicals 1992-1994”, 5th ed., Molecular Probes, Inc. (1994); Hermanson, “Bioconjugate Techniques”, Academic Press New York, N.Y. (1995); Kay M. et al., 1995. Biochemistry 34:293; Stubbs et al., 1996. Biochemistry 35:937; U.S. Pat. No. 6,350,466 to Targesome, Inc.; U.S. Pat. No. 6,037,137 to Oncoimmunin Inc.]. For specific guidance regarding conjugating of a DNA molecule, such as a DNA analyte strand of the present invention, with a fluorophore in the context of hybridization microarray applications, such as the method of the present invention, refer, for example, to Richter et al., 2002. Biotechniques 33(3):620.

Alternately, the analyte strand may be conjugated with a label such as a radioactive atom (“radiolabel”; for example, 3-hydrogen, 125-iodine, 35-sulfur, 14-carbon, or 32-phosphorus), an enzyme which catalyzes a reaction resulting in a chromogenic substrate, (“enzymatic label”), colloidal gold, or any other suitable detectable label. For a detailed review of methods of conjugating a polynucleotide, such as the analyte strand, with a suitable detectable label for practicing the method of the present invention, and for detecting such labels in the context of the present invention, refer, for example, to Laboratory Techniques in Biochemistry and Molecular Biology, Vol. 24: Hybridization With Nucleic Acid Probes, P. Tijssen, ed. Elsevier, N.Y., (1993). Detectable labels suitable for use in the present invention include any composition detectable by spectroscopic, photochemical, biochemical, immunochemical, electrical, optical or chemical means. Patents teaching the use of suitable detectable labels include U.S. Pat. Nos. 3,817,837; 3,850,752; 3,939,350; 3,996,345; 4,277,437; 4,275,149; and 4,366,241.

Examples of suitable enzymatic detectable labels for practicing the method of the present invention include horseradish peroxidase (HRP) beta-galactosidase, and alkaline phosphatase (AP). Ample guidance for suitably obtaining and utilizing enzymatic detectable labels for practicing the method of the present invention is provided in the literature of the art (for example, refer to: Khatkhatay M I. and Desai M., 1999. J Immunoassay 20:151-83; Wisdom G B., 1994. Methods Mol Biol. 32:433-40; Ishikawa E. et al., 1983. J Immunoassay 4:209-327; Oellerich M., 1980. J Clin Chem Clin Biochem. 18:197-208; Schuurs A H. and van Weemen B K., 1980. J Immunoassay 1:229-49).

Depending on the application and purpose, the analyte strand may be conjugated with the label during any of the various stages of the method of the present invention, and via any of various means well known to those of skill in the art.

Preferably, the analyte strand is conjugated with the label prior to exposure of the probe set to the analyte strand.

For conjugating the analyte strand with the detectable label prior to the exposure step of the method of the present invention, the detectable label is preferably conjugated with the analyte strand via at least one nucleotide base of the analyte strand which is suitably modified so as to be conjugatable with the detectable label.

The base modification is preferably one enabling covalent conjugation of the modified base with the detectable label. Preferably, the modified base is 5-(3-aminoallyl)-2′-deoxyuridine 5′ triphosphate (AA-dUTP). Alternately, any other suitable modified base may be employed for such covalent conjugation.

The modified base is preferably incorporated into the analyte strand during polymerization synthesis of the analyte strand (for example, during reverse transcription in the case of a cDNA analyte strand of the present invention, or during PCR amplification of an analyte strand of the present invention).

As is described and illustrated in the Examples section below, incorporating AA-dUTP into a cDNA analyte strand of the present invention during reverse transcription synthesis thereof followed by chemical conjugation of Cy5 to the modified base can be used to produce an analyte strand of the present invention which is covalently conjugated to Cy5, and which can be employed to effectively practice the method of the present invention.

Alternatively, the analyte strand may be conjugated with the label following hybridization of the analyte strand with a probe molecule of the present invention. Such post hybridization conjugation may be conveniently achieved via any of various methods well known to the skilled artisan, for example, by performing the exposure step of the method of the present invention using a biotinylated analyte strand of the present invention followed by labeling of the probe molecule hybridized analyte strand with an avidin coupled detectable label.

Any of various methods may be employed for detecting hybridization of a probe molecule of the present invention with a target analyte strand of the present invention, depending on the application and purpose.

As described hereinabove, the analyte strand is preferably conjugated with a detectable label of the present invention so as to enable measurement of hybridization thereof with a probe molecule of the present invention.

Means of detecting a detectable label of the present invention are well known to those of skill in the art [refer, for example to: Biochemistry and Molecular Biology, Vol. 24: Hybridization With Nucleic Acid Probes, P. Tijssen, ed. Elsevier, N.Y., (1993); and U.S. Pat. Nos. 3,817,837; 3,850,752; 3,939,350; 3,996,345; 4,277,437; 4,275,149; and 4,366,241]. For example, a fluorophore detectable label may be detected using a photodetector to detect emitted light, a radioactive detectable label may be detected using photographic film or a scintillation counter, an enzymatic detectable label may be detected by exposing the enzyme label to its substrate and detecting the reaction product produced by the action of the enzyme on the substrate, and a colloidal gold label may be detected by measuring light scattering thereby.

Preferably, a fluorophore detectable label of the present invention is detected according to the guidelines described in the Examples section which follows.

Preferably, the measurement step of the method of the present invention is effected by measuring a collective hybridization of the analyte strand with each distinct probe molecule of each distinct subset of probe molecules of a group of distinct subsets of probe molecules of the probe set.

Thus, data derived from detection of the detectable label following the exposure step of the method provides a measure of the hybridization of analyte strands of the analyte strand sample with a probe molecule or distinct subset of probe molecules of the present invention.

As described hereinabove, when using the method for typing the specificity repertoire of the antigen receptor chain of an individual, the specificity repertoire may be qualified by comparison to a reference specificity pattern which correlates with a phenotype related to an antigen associated disease so as to optimally qualify the individual with respect to the phenotype, and hence to optimally enable medical management of the disease in the individual. Furthermore, as described hereinabove, when using the method for typing the specificity repertoire of the antigen receptor chain in a group of individuals sharing a phenotype related to an antigen associated disease, the method of the present invention optimally enables de novo identification of such a reference specificity pattern which correlates with such a disease.

Numerous patterns of antigen receptor chain specificities are known to correlate with phenotypes related to wide range of different types of antigen associated diseases. For example, the occurrence of T-lymphocytes whose T-cell receptors include specific Vbeta segments is associated with occurrence of diseases such as: malignant diseases, including various types of CML (Zhang Y P. et al., 2002. Zhongguo Shi Yan Xue Ye Xue Za Zhi. 10:122-5), and non Hodgkin's lymphoma (Lu Y H. et al., 2002. Zhongguo Shi Yan Xue Ye Xue Za Zhi. 10:119-21); infectious diseases such as human immunodeficiency virus (HIV) induced acquired immunodeficiency syndrome (AIDS; Marchalonis J J. et al., 1997. Clin Immunol Immunopathol. 82:174-89), chronic human Chagas' disease (Fernandez-Mestre M T. et al., 2002. Tissue Antigens 60:10-5), chronic hepatitis C (Kashii Y. et al., 1997. J Hepatol. 26:462-70), otitis media (Takeuchi K. et al., 1996. Ann Otol Rhinol Laryngol. 105:213-7) and chronic hepatitis B (Dou H Y. et al., 1998. J Biomed Sci. 5:428); autoimmune diseases such as rheumatoid arthritis (Zhang Z. et al., 2002. Chin Med J (Engl). 115:856; Osman G E. et al., 1999. Immunogenetics 49:764-72), myasthenia gravis (Navaneetham D. 1998. J Autoimmun. 11:621-33), lupus nephritis (Sutmuller M. et al., 1998. Immunology 95:18), Sjogren's syndrome (Yamamoto T. et al., 1998. Eur J Dermatol. 8:248), IgA nephropathy (Muro K. et al., 2002. Nephron 92:56-63) and autoimmune hepatitis (Arenz M. et al., 1998. J Hepatol. 28:70); allergic diseases such as hypersensitivity pneumonitis (Trentin L. et al., 1997. Am J Respir Crit Care Med. 155:587); inflammatory diseases such as sarcoidosis (Trentin L. et al., 1997. Am J Respir Crit Care Med. 155:587), primary biliary cirrhosis (Mayo M J. et al., 1996. Hepatology 24:1148-55; Ohmoto M. et al., 1997. Hepatology 25:33-7), Takayasu's arteritis (Seko Y. et al., 1996. Circulation 93:1788), hemophagocytic lymphohistiocytosis (Nagano M. et al., 1999. Blood 94:2374-82), and inclusion body myositis (Fyhr I M. et al., 1998. J Neuroimmunol. 91:129-34); and transplantation related diseases such as alloreactivity to defined HLA-DR alleles (Lobashevsky A. et al., 1996. Transplantation. 62:1332), cardiograft rejection (Oaks et al., 1995. Am J Med Sci. 309:26-34), and porcine xenograft rejection in humans (Chen M. et al., 1999. Transplantation 68:586-9).

Furthermore, numerous patterns of antigen receptor chain specificities are known to correlate with occurrence of tumor infiltrating lymphocytes (TILs) associated with various malignancies. For example, the occurrence of T-lymphocytes whose T-cell receptor beta chains include specific Vbeta segments is associated with occurrence of tumor infiltrating lymphocytes in malignancies such as oral squamous cell carcinoma (Mouri T. et al., 1996. Cancer Immunol Immunother. 43:10-8), colorectal tumors (Ostenstad B. et al., 1994. Br J Cancer. 69:1078-82), renal cell carcinoma (Gaudin C. et al., 1995. Cancer Res. 55:685-90), primary gastric malignant B-cell lymphoma (Yumoto N. et al., 1995. Virchows Arch. 426:11-8), nasopharyngeal carcinoma (Chen Y. et al., 1995. Br J Cancer. 72:117-22), Yamamoto Y. et al., 1993. Cancer Immunol Immunother. 37:163-8), metastatic melanoma (Willhauck M. et al., 1996. Clin Cancer Res. 2:767-72; Zeuthen J. et al., 1995. Arch Immunol Ther Exp (Warsz). 43:123-33), and head and neck cancer (Chikamatsu K. et al., 1994. Jpn J Cancer Res. 85:626-32; Caignard A. et al., 1994. Cancer Res. 54:1292-7).

Hence, since as described in the Examples section which follows, the method of the present invention can be used for optimally typing a human individual's repertoire of T-cell receptor beta chain specificities in terms of specific combinations of at least two variable segments including a Vbeta segment, and since as described hereinabove, the occurrence of specific T-cell receptor Vbeta segments correlating with numerous diseases is well characterized, including for infectious, autoimmune, allergic, transplantation related, malignant, and inflammatory diseases, the method of the present invention can be used, for example, for optimally diagnosing such antigen associated diseases in a human individual. One of ordinary skill in the art will possess the necessary expertise for applying the teachings of the present invention towards diagnosis of such diseases in light of the ample art literature available, such as listed hereinabove, regarding the prevalence of specific antigen receptor chain variable region segments which correlate with such diseases, and in light of the teachings of the present invention.

In the case of malignancies, the tumor infiltrating lymphocytes described hereinabove expressing specific T-cell receptor Vbeta segments are widely understood as mediating anti-tumor immunity. Hence, the method of the present invention can be used to identify tumor infiltrating lymphocytes having anti-cancer activity, which cells can be expanded ex-vivo and reinfused in the context of anti-cancer cell therapy. One of ordinary skill in the art will possess the necessary expertise for utilizing the teachings of the present invention to perform such anti-cancer adoptive cell therapy.

Specific T-cell receptor Vbeta usage has been shown to correlate with anti-human immunodeficiency virus (HIV) immune responses in individuals immunized according to various regimens (Pancre V. et al., 2002. Clin Exp Immunol. 129:429-37; Guzman C A. et al., 1988. Eur J Immunol. 28:1807-14). As such, the method of the present invention can be used for monitoring responses to therapy for diseases such as acquired immunodeficiency syndrome (AIDS) caused by HIV.

As described hereinabove, a novel reference specificity pattern of the present invention which correlates with a phenotype related to an antigen associated disease may be identified by suitably analyzing the antigen receptor chain specificity repertoire of a number of individuals which share a phenotype associated with the disease. One of ordinary skill in the art will possess the necessary expertise for applying the teachings of the present invention towards diagnosis of antigen associated diseases, such as those listed above, in light of the ample art literature available, such as listed hereinabove, regarding the prevalence of specific antigen receptor chain variable region segments which correlates with such diseases. One of ordinary skill in the art will furthermore possess the necessary expertise for analyzing specificity repertoires of an antigen receptor chain, such as specificity repertoires of the present invention, so as to identify therein a reference specificity repertoire which correlates with a phenotype related to an antigen associated disease. Numerous tools are available to the ordinarily skilled artisan for effecting such specificity pattern recognition or pattern comparison. Such tools include various computer programs, including those employing support vector machines, fuzzy logic algorithms, artificial neural networks, principle component analysis, expert systems, clustering algorithms, and/or other pattern recognition algorithms. Ample guidance for effecting the above described specificity pattern recognition or pattern comparison applications of the method of the present invention is available in the literature of the art (refer, for example, to: Hariharan R., 2003. Pharmacogenomics 4:477; and Dudoit et al. Biotechniques 2003, March Suppl:45-51; WIPO Application No. WO0208755A2; Azuaje F., 2003. Brief Bioinform. 4:31; and Valafar, 2002. Ann N Y Acad. Sci. 980:41). It will be appreciated that since the T-cell receptor is MHC restricted it may be advantageous to stratify specificity patterns specific to T-cell receptor chains according to the genetic MHC background. The MHC genes of an individual can be classified by conventional methods like serum analysis with antibodies, PCR analysis using appropriate primer or by DNA array MHC analysis using appropriate oligonucleotide probes.

Therefore, the method can be used adapted for optimizing various aspects of the medical management of essentially any of the vast range of antigen associated diseases, of which selected examples are provided hereinbelow.

Examples of autoimmune diseases associated with antibody mediated immune responses include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad. Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost. 2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like beta-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefiuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Examples of diseases associated with T cell mediated autoimmune diseases, include, but are not limited to, rheumatoid diseases, rheumatoid arthritis (Tisch R, McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann N Y Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of antigen associated diseases associated with antigen specific delayed type hypersensitivity include, but are not limited to, contact dermatitis and drug eruption.

Examples of organ/tissue specific autoimmune diseases include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

Examples of antigen specific infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Examples of antigen specific transplantation related diseases, but are not limited to, graft rejection, chronic graft rejection, subacute graft rejection, hyperacute graft rejection, acute graft rejection and graft versus host disease.

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

Examples of antigen specific inflammatory diseases include, but are not limited to; inflammation associated with injuries, neurodegenerative diseases, ulcers, prosthetic implants, menstruation, septic shock, anaphylactic shock, toxic shock syndrome, cachexia, necrosis and gangrene; musculo-skeletal inflammations, idiopathic inflammations.

Thus, the present invention enables optimal typing of an antigen receptor chain specificity repertoire of a human individual, and, as such, can be used to optimally enable medical management of a disease associated with an antigen specific protective or pathogenic immune response.

It is expected that during the life of this patent many relevant medical diagnostic techniques will be developed and the scope of the term “typing” is intended to include all such new technologies a priori.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### Example 1

### Repertoire Scale Typing of Human TCRβ Rearranged Variable Region Segment Combinations

Background: Diseases associated, with a protective or pathogenic antigen specific immune response, such as infectious, autoimmune, allergic, transplantation related, malignant and inflammatory diseases, include numerous highly debilitating and/or lethal diseases whose medical management is suboptimal, for example, with respect to prevention, diagnosis, treatment, patient monitoring, prognosis, and/or drug design. Optimal performance of such aspects of medical management of such a disease in an individual would be enabled by a method of optimally typing an antigen receptor chain specificity repertoire of the individual. Such typing could be used to optimally qualify the antigen receptor specificity repertoire of the individual with respect to a reference specificity pattern correlating with a phenotype associated with the disease. Such qualification could be used to optimally qualify the individual with respect to the phenotype, and hence to facilitate optimal performance of such aspects of medical management of such a disease in the individual. Such a typing method could further be used to enable identification of novel reference specificity patterns shared among individuals sharing a phenotype associated with such a disease. While various methods of typing antigen receptor chain specificity repertoires have been proposed in the prior art, these have been distinctly suboptimal for numerous reasons, as described above. Thus, in order to overcome such prior art limitations, the present inventors have devised and successfully tested a novel and optimal method of typing antigen receptor chain specificities, as described below.

Materials and Methods:

Design of Oligonucleotide Hybridization Probe Set:

The presently described strategy for typing a repertoire of human TCRβ variable region segment combinations is based on a DNA oligonucleotide microarray utilizing only 299 degenerate probes (pooled probe sets) each of which enabling specific detection of a set of target cDNA sequences of rearranged TCRβ CDR3 regions each of which corresponding to one of 23 novel Vβ-segment groups conceived by the present inventors and one of 13 possible Jβ-segments. Namely, while conceiving the present invention, it was unexpectedly uncovered by the present inventors that Vβ-segments could be conveniently grouped into as few as 23 novel groups each of which having shared CDR3 specific amino acid sequences, thereby enabling design of oligonucleotide probes suitable for optimally typing human TCRβ variable region segment combination repertoires.

Each degenerate probe of the set of 299 degenerate probes is a degenerate DNA sequence composed of all possible combinations of the following modules, from 5′ to 3′:

(i) one of 23 consensus DNA sequences (some of which degenerate) each of which encoding a CDR3 specific carboxy terminal portion of a Vβ-segment belonging to one of 23 novel V-segment groups (see Table 1, below);

(ii) the degenerate consensus DNA sequence 5′-gggac(a/t)(a/g)_{g}(c/g)gg(c/g)-3′ (SEQ ID NO: 47) which includes all known sequences encoding a Dβ-segment; and

(iii) one of 13 sequences each of which encoding the CDR3 specific portion of one of the 13 Jβ-segments (see Table 2, below). This results in a total set of 299 degenerate probes, according to the calculation: [23 degenerate Vβ-segment specific probe segments]×[1 degenerate Dβ-segment specific probe segment]×[13 Jβ-segment specific probe segments]=299 degenerate probes.

Table 3 lists a representative degenerate probe subset, each degenerate probe of which being specific for cDNAs of a variable region which includes a Vβ-segment belonging to novel Vβ-segment group No. 1 (see Table 1), and which includes one of the 13 possible Jβ-segments.

TABLE 1
Novel Vβ-segment groups and sequences of Vβ-segment specific modules of probes.

| | CDR3 specific | | |
|---|---|---|---|
| | amino acid | | Consensus DNA sequence |
| Novel Vβ- | sequence of | | encoding carboxy terminal |
| segment | group members | Vβgenes encoding Vβ-segments | portion of Vβ-segments |
| group No. | (1-letter code) | belonging to novel group* | belonging to novel group |
| 1 | C | BV12S1A1N1, BV12S1A1N4 | 5′-gtgtacttctgt-3′ |
| | (SEQ ID NO:1) | | (SEQ ID NO:24) |
| 2 | CA | BV1S1A2, BV12S1A1N3 | 5′-tatttctgtgcc-3′ |
| | (SEQ ID NO:2) | | (SEQ ID NO:25) |
| 3 | CS | BV4S1A2T | 5′-tatctctgcagc-3′ |
| | (SEQ ID NO:3) | | (SEQ ID NO:26) |
| 4 | CAS | BV6S4A5N1T, BV6S4A5N2T, | 5′-(c/t)t(c/t)tg(c/t)gccagc-3′ |
| | (SEQ ID NO:4) | BV7S3A1T, BV13S6A4T, | (SEQ ID NO:27) |
| | | BV13S2A3PT, BV9S1A2T, | |
| | | BV5S1A1T | |
| 5 | CAW | BV20S1A1N3 | 5′-ctctgtgcctgg-3′ |
| | (SEQ ID NO:5) | | (SEQ ID NO:28) |
| 6 | CSA | BV2S1A2, BV2S1A3N3T, | 5′-atctgcagtgct-3′ |
| | (SEQ ID NO:6) | BV2S2A1O, BV2S1A4T, | (SEQ ID NO:29) |
| | | BV2S1A3N2T | |
| 7 | CASS | BV16S1A1N2, BV6S2A1N2T, | 5′-tg(c/t)gccag(c/t)ag(c/t)-3′ |
| | (SEQ ID NO:7) | BV6S3A1N2T, BV6S6A1T, | (SEQ ID NO:30) |
| | | BV6S4A2, BV6S4A6T, BV21S2A3T, | |
| | | BV21S2A1N1, BV21S2A1N2, | |
| | | BV21S3A1T, BV21S3A2N1, | |
| | | BV25S1A3T, BV23S1A1T, | |
| | | BV5S6A3N1T, BV1S1A1N2T, | |
| | | BV5S6A2T, BV5S6A1T, BV5S3A1T, | |
| | | BV5S3A3T, BV5S4A1T, | |
| | | BV9S2A1PT, BV7S2A1N2T, | |
| | | BV13S2A2PT, BV8S2A2N2T, | |
| | | BV22S1A2N2, BV22S1A1T, | |
| | | BV13S6A3T, BV13S6A1N1, | |
| | | BV13S6A1N2, BV11S1A2T, | |
| | | BV7S2A2T, BV7S2A1N1T, | |
| | | BV7S2A1N3T, BV6S4A4T, | |
| | | BV6S1A1N2T, BV17S1A3T, | |
| | | BV17S1A2T, BV12S2A3T | |
| 8 | CATS | BV24S1A2T, BV245S1A1T | 5′-tgtgccaccagc-3′ |
| | (SEQ ID NO:8) | | (SEQ ID NO:31) |
| 9 | CAWS | BV20S1A3T, BV20S1A2P, | 5′-tgtgcctggagt-3′ |
| | (SEQ ID NO:9) | BV20S1AIN1, BV20S1A1N2 | (SEQ ID NO:32) |
| 10 | CSVE | BV4S1A3T, BV4S1A1T, BV4S2O | 5′-tgcagcgttgaa-3′ |
| | (SEQ ID NO:10) | | (SEQ ID NO:33) |
| 11 | CSAR | BV2S1A3N1, BV2S1A5T, BV2S1A1, | 5′-tgcagtgctaga-3t |
| | (SEQ ID NO:11) | BV2S2A2O | (SEQ ID NO:34) |
| 12 | CASSL | BV8S2A2N1, BV8S2A1T, BV8S1, | 5′-tg(c/t)gccagcag(c/t)tt(a/g)-3′ |
| | (SEQ ID NO:12) | BV14S1, BV3S1, BV6S1A1N1, | (SEQ ID NO:35) |
| | | BV6S1A3T, BV6S5A1N2T, | |
| | | BV6S8A2T, BV6S2A1N1, | |
| | | BV6S2A2T, BV6S6A2T, | |
| | | BV6S3A1N1, BV6S4A1, BV6S4A3T, | |
| | | BV6S5A1N1, BV6S5A2, BV21S1, | |
| | | BV21S2A2, BV21S3A2N2, | |
| | | BV23S1A2T, BV5S1A1T, | |
| | | BV5S6A3N2T, BV5S7P, BV5S3A2T, | |
| | | BV5S2, BV5S4A2T | |
| 13 | CASSQ | BV19S1P, BV16S1A1N1, | 5′-tg(c/t)gccagcagccaa-3′ |
| | (SEQ ID NO:13) | BV25S1A1T, BV25S1A2PT, | (SEQ ID NO:36) |
| | | BV9S1A1T, BV9S2A2PT, | |
| | | BV7S2A1N4T, BV7S3A2T, | |
| | | BV7S1A1N1T, BV7S1A1N2T | |
| 14 | CASSY | BV13S2A1T, BV13S6A2T, BV13S8P, | 5′-tgtgccagcagtta(c/t)-3′ |
| | (SEQ ID NO:14) | BV13S7, BV13S4, BV13S1 | (SEQ ID NO:37) |
| 15 | CASSE | BV11S1A1T, BV11S2OP, BV13S3, | 5′-tgtgccagcagtga(a/g)-3′ |
| | (SEQ ID NO:15) | BV22S1A2N1, BV12S3, BV12S2A1T | (SEQ ID NO:38) |
| 16 | CASSV | BV1S1A1N1 | 5′-tgtgccagcagcgta-31 |
| | (SEQ ID NO:16) | | (SEQ ID NO:39) |
| 17 | CASSI | BV17S1A1T | 5′-tgtgccagtagtata-3′ |
| | (SEQ ID NO:17) | | (SEQ ID NO:40) |
| 18 | CASSD | BV13S5 | 5′-tgtgccagcagtgac-3′ |
| | (SEQ ID NO:18) | | (SEQ ID NO:41) |
| 19 | CASSP | BV18S1 | 5′-tgtgccagctcacca-3′ |
| | (SEQ ID NO:19) | | (SEQ ID NO:42) |
| 20 | CASGL | BV8S3 | 5′-tgtgctagtggtttg-3′ |
| | (SEQ ID NO:20) | | (SEQ ID NO:43) |
| 21 | CATSR | BV24S1A3T | 5′-tgtgccaccagcaga-3′ |
| | (SEQ ID NO:21) | | (SEQ ID NO:44) |
| 22 | CAISE | BV12S1A1N2 | 5′-tgtgccatcagtgag-3′ |
| | (SEQ ID NO:22) | | (SEQ ID NO:45) |
| 23 | CATSDL | BV15S1, BV15S2OP | 5′-tgtgccaccagtgatttg-3′ |
| | (SEQ ID NO:23) | | (SEQ ID NO:46) |

* according to Arden nomenclature (Arden, B. et al., 1995. Immunogenetics 42,455-500)

TABLE 2

DNA sequences encoding CDR3 specific N-terminal
portions of Jβ-segments: Jβ-segment specific
modules of probes.

| | DNA sequence of CDR3 specific N- |
|---|---|
| Jβ-segment | terminal portion of Jβ-segment |
| 1.1 | 5′-actgaagctttc-3′ |
| | (SEQ ID NO:48) |
| 1.2 | 5′-tatggctacacc-3′ |
| | (SEQ ID NO:49) |
| 1.3 | 5′-ggaaacaccatatat-3′ |
| | (SEQ ID NO:50) |
| 1.4 | 5′-aatgaaaaactgttt-3′ |
| | (SEQ ID NO:51) |
| 1.5 | 5′-aatcagccccagcat-3′ |
| | (SEQ ID NO:52) |
| 1.6 | 5′-tataattcacccctccac-3′ |
| | (SEQ ID NO:53) |
| 2.1 | 5′-tacaatgagcagttc-3′ |
| | (SEQ ID NO:54) |
| 2.2 | 5′-accggggagctgttt-3′ |
| | (SEQ ID NO:55) |
| 2.3 | 5′-acagatacgcagtat-3′ |
| | (SEQ ID NO:56) |
| 2.4 | 5′-aaaaacattcagtac-3′ |
| | (SEQ ID NO:57) |
| 2.5 | 5′-gagacccagtac-3′ |
| | (SEQ ID NO:58) |
| 2.6 | 5′-ggggccaacgtcctgact-3′ |
| | (SEQ ID NO:59) |
| 2.7 | 5′-tacgagcagtac-3′ |
| | (SEQ ID NO:60) |

TABLE 3

Representative probe set: degenerate probe
sequences of degenerate probes having a
Vβ-segment specific module specific for Vβ-
segments belonging to β-segment group No.
1 (CDR3 specific amino acid sequence = Cys
residue)

| Jβ gene | |
|---|---|
| specificity | |
| of probe | DNA sequence of probe* |
| 1.1 | 5′-gtgtacttctgtgggac(a/t)(a/g)g(c/g)gg |
| | (c/g)actgaagctttc-3′ |
| | (SEQ ID NO:61) |
| 1.2 | 5′-gtgtacttctgtgggac(a/t)(a/g)g(c/g)gg |
| | (c/g)tatggctacacc-3′ |
| | (SEQ ID NO:62) |
| 1.3 | 5′-gtgtacttctgtgggac(a/t)(a/g)g(c/g)gg |
| | (c/g)ggaaacaccatatat-3′ |
| | (SEQ ID NO:63) |
| 1.4 | 5′-gtgtacttctgtgggac(a/t)(a/g)g(c/g)gg |
| | (c/g)aatgaaaaactgttt-3′ |
| | (SEQ ID NO:64) |
| 1.5 | 5′-gtgtacttctgtgggac(a/t)(a/g)g(c/g)gg |
| | (c/g)aatcagccccagcat-3′ |
| | (SEQ ID NO:65) |
| 1.6 | 5′-gtgtacttctgtgggac(a/t)(a/g)g(c/g)gg |
| | (c/g)tataattcacccctccac-3′ |
| | (SEQ ID NO:66) |
| 2.1 | 5′-gtgtacttctgtgggac(a/t)(a/g)g(c/g)gg |
| | (c/g)tacaatgagcagttc-3′ |
| | (SEQ ID NO:67) |
| 2.2 | 5′-gtgtacttctgtgggac(a/t)(a/g)g(c/g)gg |
| | (c/g)accggggagctgttt-3′ |
| | (SEQ ID NO:68) |
| 2.3 | 5′-gtgtacttctgtgggac(a/t)(a/g)g(c/g)gg |
| | (c/g)acagatacgcagtat-3′ |
| | (SEQ ID NO:69) |
| 2.4 | 5′-gtgtacttctgtgggac(a/t)(a/g)g(c/g)gg |
| | (c/g)aaaaacattcagtac-3′ |
| | (SEQ ID NO:70) |
| 2.5 | 5′-gtgtacttctgtgggac(a/t)(a/g)g(c/g)gg |
| | (c/g)gagacccagtac-3′ |
| | (SEQ ID NO:71) |
| 2.6 | 5′-gtgtacttctgtgggac(a/t)(a/g)g(c/g)gg |
| | (c/g)ggggccaacgtcctgact-3′ |
| | (SEQ ID NO:72) |
| 2.7 | 5′-gtgtacttctgtgggac(a/t)(a/g)g(c/g)gg |
| | (c/g)tacgagcagtac-3′ |
| | (SEQ ID NO:73) |
| *Bold nucleotides denote degenerate sequence of Dβ-segment specific module (SEQ ID NO:47), nucleotides to the left of the Dβ-segment specific module denote the sequence of the Vβ-segment specific module specific for Vβ-segments belonging to Vβ-segment group No. 1 (SEQ ID NO:24; CDR3 specific consensus amino acid sequence is a Cys residue; see Table 1), and nucleotides to the right of the Dβ-segment specific module denote the sequence of the Jβ-segment specific module (SEQ ID | |
| #48-60; see Table 2). | |

Preparation of TCRβ variable region cDNA of an individual: Total RNA from 5×10⁸ peripheral blood mononuclear cells (PBMCs) obtained from a healthy individual was purified using RNeasy Maxi Kit (QIAGEN). To produce TCRβ variable region target cDNA, aliquots of 100 micrograms of total RNA were reverse-transcribed using the specific MBC2 primer 5′-TGCTTCTGATGGCTCAAACACAGCGACCT-3′ (SEQ ID NO: 74). The RNA was incubated with 3 microliters of MBC2 primer (100 micromolar) in a 100 microliter reaction mixture at 70 degrees centigrade for 10 minutes, then snap-frozen in a dry ice/ethanol bath. The annealed primer-RNA mixture was then supplemented with 20 microliters of 5× amplification buffer, 10 microliters 0.1 M dithiothreitol (DTT), 2 microliters of dNTPs mixture (25 mM each), including 5-(3-aminoallyl)-2′-deoxyuridine 5′ triphosphate (AA-dUTP) at an AA-dUTP to dTTP ratio of 1:1 (AA-dUTP to be subsequently labeled by Cy5 fluorochrome), 2 microliters of RNaseOUT (Invitrogen) and 7 microliters of SuperScript-II Reverse Transcriptase (Invitrogen), and the reaction mixture was incubated at 42 degrees centigrade for 3 hours. To hydrolyze RNA, 33 microliters of 1 M NaOH and 33 microliters of 0.5 M EDTA were added and the reaction mixture was incubated at 65 degrees centigrade for 15 minutes, followed by addition of 33 microliters of 1 M HCl for neutralization. Unincorporated AA-dUTP and free amines were removed from the reaction mixture by using a modified protocol of QIAGEN PCR purification kit: 1 ml of buffer PB were added to the reaction, which was then loaded on a QIAquick column. The column washed twice with 750 microliters of phosphate wash buffer (5 mM KPO₄ pH 8.0, 80% ethanol) and dried by additional microcentrifugation for 1 minute at maximal speed. The cDNA was eluted twice with 30 microliters of phosphate elution buffer (4 mM KPO₄, pH 8.5), yielding a total elution volume of 60 microliters.

Preparation of target cDNA of a specific (clonal) human TCRβ chain: Complementary DNA of a specific TCRβ chain including a Jβ2.1-segment and a Vβ-segment belonging to novel Vβ-segment group 4 (having a Vβ-segment specific portion of CDR3 consisting of a CAS amino acid sequence, see Table 1, above) cloned in vector pGEM-T-Easy (Promega) was amplified by PCR using the M13 universal primers (Promega). The dNTPs mixture included AA-dUTP at an AA-dUTP to dTTP molar ratio of 4:1. The PCR product was purified from unincorporated AA-dUTP and free amines, as described above, in a final volume of 60 microliters.

Fluorescent labeling of target cDNA: The target cDNA pool and target clonal cDNA were dried in a speed-vac and resuspended in 5.5 microliters of freshly prepared 0.1 M Na₂CO₃ buffer, pH 9.0. Aliquots of 5.5 microliters of Cy5 ester dissolved in dimethylsulfoxide (DMSO) were added to the mixture, and the mixture was incubated for 1 hour in the dark at room temperature. Following the dye-coupling reaction, 43 microliters of 0.1 M sodium acetate, pH 5.2 was added to the mixture and uncoupled dye was removed using QIAGEN PCR purification kit according to the manufacturer's instructions. The labeled cDNA was eluted twice with 30 microliters of EB buffer to obtain a final elution volume of 60 microliters.

For determination of labeling efficiency, labeled target cDNA was assayed for absorbance at 260 nm and 650 nm, and total cDNA and Cy5 content was calculated according to the following equations:
pmol clonal cDNA=[OD260×volume(microliters)×50(ng/microliter)×1000 (pg/ng)]/324.5 (g/pmol)
pmol cDNA pool=[OD260×volume(microliters)×37(ng/microliter)×1000 (pg/ng)]/324.5 (pg/pmol)
pmol Cy5=[OD650×volume(microliters)]/0.25
DNA/Cy5 molar ratio=pmol DNA/pmol Cy5

Samples containing more than 200 pmol of dye incorporation per probe and a ratio of less than 50 DNA molecules per dye molecule were selected for hybridization.

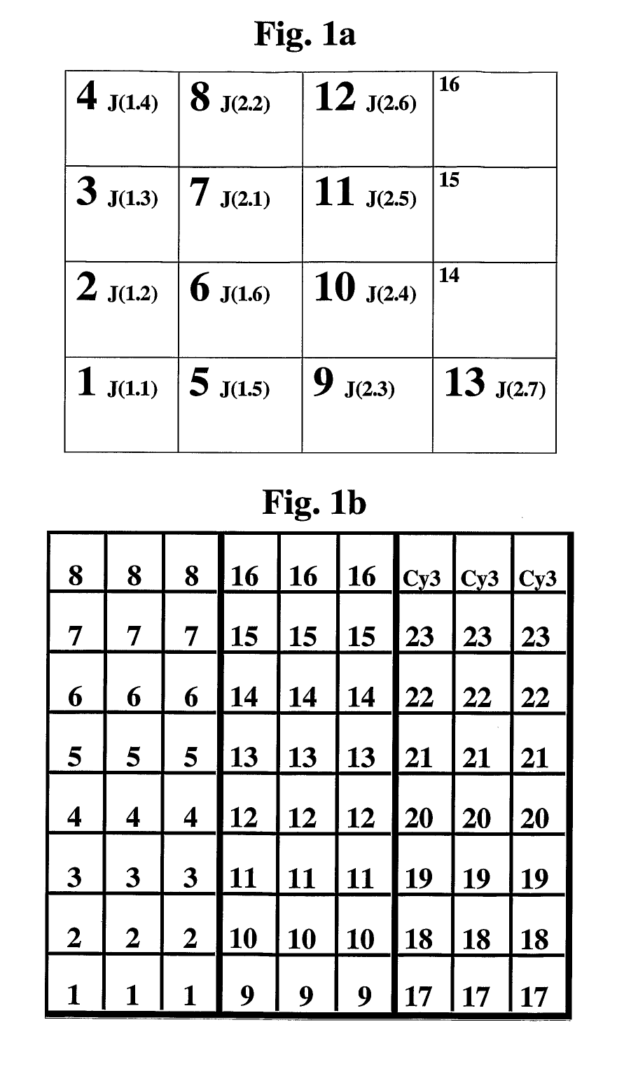

Microarray printing: Each of the 299 degenerate oligonucleotide probes (see Tables 1-3, above) was diluted to a final concentration of 10 micromolar with addition of DMSO to a final concentration of 50% DMSO, in a 384-well plate. The probes were printed in triplicate on SuperAmine slides (ArrayIt, TeleChem), using a Total Array System (TAS) robot (BioRobotics) with a solid 16-pin-head tool. Dot center-to-center distance was set to 400 microns. Out of the 16 subarrays of the chip, 13 were each printed with the set of probes specific for cDNA of TCRβ variable regions including one of the 13 Jβ-segments (FIG. 1a). Within each subarray, each of 23 sets of cell triplicates were printed with the degenerate probe specific for cDNAs of TCRβ, variable regions including the Jβ-segment specific to the subarray and including a Vβ-segment belonging to one of the 23 novel Vβ-segment groups (FIG. 1b). Printed slides were stored clean in a dark box prior to use.

Hybridization of labeled target cDNA to the microarray: Printed slides were incubated in preheated prehybridization buffer (5×SSC, 0.1% sodium dodecyl sulfate [SDS], 1% BSA) at 42 degrees centigrade for 45 minutes, washed twice in 100 ml MilliQ column-purified water and dried by centrifugation in a slide-box underlayed with Whatman paper for liquid absorption. Slides were used immediately following prehybridization treatment. Depending on labeling efficiency, 200-500 ng samples of target clonal cDNA or of cDNA pool samples were dried in a speed-vac and resuspended in 12 microliters hybridization buffer (50% formamide, 5×SSC, 0.1% SDS). Target cDNAs were denatured at 95 degrees centigrade for 3 minutes, snap-frozen on ice for 30 seconds, centrifuged for 1 minute, and immediately applied to the printed area of the slide. This was followed by overlaying of the printed area of the slide with a cover slip to remove bubbles. The hybridization slides were sealed with foil and incubated overnight in a hybridization chamber (ArrayIt, TeleChem) at 23 degrees centigrade under low stringency conditions. Low stringency hybridization conditions are employed in order to obtain a probe-target hybridization level yielding a distinctive hybridization pattern characteristic of an individual's TCRβ variable region repertoire. Following hybridization, the slides were washed three times in 250 ml of wash buffers (1st wash buffer, 1×SSC, 0.1% SDS; 2nd wash buffer, 1×SSC; 3rd wash buffer, 0.1×SSC) for 4 minutes with slow shaking. After the washes, the slides were dipped three times in MilliQ column-purified water and dried by centrifugation in a slide-box underlayed with Whatman paper for liquid absorption. The dried slides were then laser-scanned for Cy5 and Cy3 detection using a ScanArray 4000XL scanner (GSI-Lumonics).

Experimental Results:

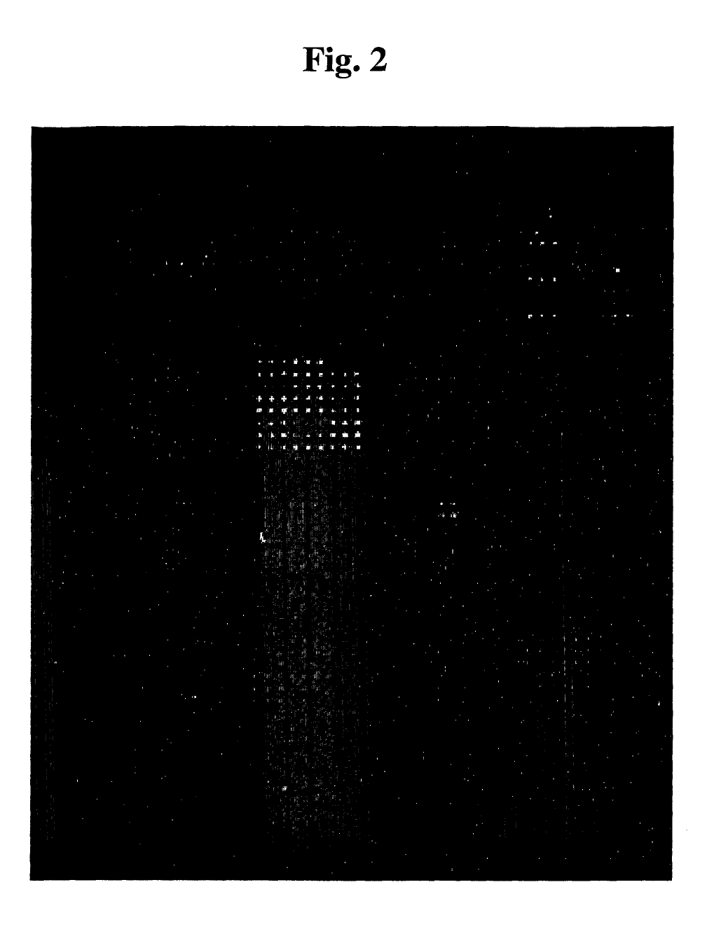

The TCR chip enables accurate characterization of Jβ and Vβ gene segment specificity of clonal cDNA of a specific human TCRβ chain: In order to determine the binding specificity and capacity of the microarray under the low-stringency hybridization conditions employed, the oligonucleotide microarray was used to analyze a clonal cDNA target of a specific TCRβ chain including a Jβ2.1-segment and a Vβ-segment belonging to novel Vβ-segment group No. 4 (having a Vβ-segment specific portion of CDR3 consisting of a CAS amino acid sequence motif, see Table 1). As shown in FIG. 2, scanning of the hybridized slide for Cy5 fluorescence clearly demonstrated that the target cDNA specifically hybridized with high affinity to the subarray specific for its Jβ2.1-segment, and within the Jβ2.1-segment specific subarray to cells specific for Vβ-segments Vβ4, Vβ16 and Vβ18, which all belong to novel Vβ-segment group No. 4, similarly to the target. Thus, the low stringency conditions used for the hybridization enabled characterization of the Vβ- and Jβ-segment specificity of a cDNA of a specific TCRβ variable region.

The TCR chip enables characterization of the global TCRβ rearrangement repertoire of an individual in terms of 299 novel TCRβ rearrangement groups: After optimizing the low stringency hybridization conditions, RNA was extracted from PBMCs of a specific healthy human individual, and used to produce a TCRβ variable region target cDNA pool using a specific primer. The target cDNA pool was subjected to microarray analysis, and, as can be seen in FIG. 3, the pool of target cDNAs hybridized to the oligonucleotide probes with a highly distinctive global pattern of specificities and intensities characteristic of the TCR specificity repertoire of the individual tested.

Conclusion: The above described results demonstrate that the novel method devised by the present inventors can be used for typing a specificity repertoire of an antigen receptor chain, in particular that of T-cell receptor beta chain, in a human individual. By virtue of enabling such typing using an optimally restricted set of specificities, via an optimally simplified variable region segment classification scheme, enabling classification of rearranged variants of an antigen receptor chain according to a combination of variable region segments, the method of the present invention can be used for typing an antigen receptor chain specificity repertoire with optimal convenience, rapidity, flexibility, and utility relative to all prior art methods. By virtue of enabling optimal typing of an antigen receptor chain specificity repertoire of an individual, the method of the present invention can be used for optimally qualifying such a specificity repertoire with respect to a reference specificity pattern correlating with a phenotype related to an antigen associated disease, and thereby can be used for optimally qualifying such an individual with respect to such a phenotype. Since such qualification enables optimal performance of numerous aspects of medical management of such a disease in an individual, including prevention, diagnosis, treatment, patient monitoring, prognosis, and drug design, the method of the present invention therefore enables optimal medical management of such a disease in an individual. Furthermore, the presently described typing method can be used to optimally identify a novel reference specificity pattern characteristic of a phenotype related to an antigen associated disease by virtue of enabling optimal analysis of an antigen receptor chain specificity repertoire in individuals sharing such a phenotype.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by its accession number was specifically and individually indicated to be incorporated herein by reference. In addition, citation or identification of any reference in this application shall not be construed as an admission that such reference is available as prior art to the present invention.

What is claimed is:

1. A method of typing a variable region of a specific variant of an antigen receptor chain, the method comprising:
(a) exposing a probe set to a sense or antisense strand of a polynucleotide encoding at least a portion of the variable region of the specific variant of the antigen receptor chain, wherein said probe set includes a plurality of probe molecules, wherein each probe molecule of said plurality of probe molecules is substantially complementary to a sense or antisense strand of a nucleic acid sequence region of a specific polynucleotide encoding a variant of the antigen receptor chain, said nucleic acid sequence region distinctly encoding a specific combination of at least two variable region segments of the antigen receptor chain; and
(b) measuring a hybridization of each probe molecule of said plurality of probe molecules with said sense or antisense strand of said nucleic acid sequence region of said polynucleotide encoding at least a portion of the variable region of the specific variant of the antigen receptor chain, thereby typing the variable region of the specific variant of the antigen receptor chain.

2. The method of claim 1, wherein each probe molecule of said probe set is attached to a probe array at a specific addressable location of a plurality of addressable locations included in said probe array.

3. The method of claim 2, wherein said probe array includes said plurality of addressable locations at a surface density of at least 625 specific addressable locations per square centimeter of a support comprised in said probe array.

4. The method of claim 1, wherein step (b) is effected by measuring a collective hybridization of said sense or antisense strand of said polynucleotide encoding at least said portion of the variable region of the specific variant of the antigen receptor chain, with each distinct probe molecule of each distinct subset of probe molecules of a group of distinct subsets of probe molecules of said probe set, wherein said group of distinct subsets of probe molecules includes a number of distinct subsets of probe molecules selected from a range of 1-299 distinct subsets of probe molecules.

5. The method of claim 4, wherein each distinct subset of probe molecules of said group of distinct subsets of probe molecules includes a number of distinct probe molecules selected from a range of 1-128 distinct probe molecules.

6. The method of claim 4, wherein each distinct subset of probe molecules of said group of distinct subsets of probe molecules is attached to a probe array at a specific addressable location of a plurality of addressable locations included in said probe array.

7. The method of claim 6, wherein said probe array includes said plurality of addressable locations at a surface density of at least 625 specific addressable locations per square centimeter of a support comprised in said probe array.

8. The method of claim 1, wherein the polynucleotide encoding at least said portion of the variable region of the specific variant of the antigen receptor chain is a complementary DNA molecule.

9. The method of claim 1, wherein said probe set includes a number of probe molecules selected from the group consisting of 2, 3, 4, 5, 6, 7, 8, 9, 10, 11, 12, 13, 14, 15, 16, 17, 18, 19, 20, 21, 22, 23, 24 or 25 probe molecules.

10. The method of claim 1, wherein said probe set includes a number of probe molecules selected from a range of 26-30, 31-35, 36-40, 41-45, 46-50, 51-55, 56-60, 61-65, 66-70, 71-75, 76-80, 81-85, 86-90, 91-95, 96-100, 101-150, 151-200, 201-250, 251-300, 301-350, 351-400, 401-450, 451-500, 501-550, 551-600, 601-650, 651-700, 701-750, 751-800, 801-850, 851-900, 901-950, 951-1,000, 1,001-1,100, 1,101-1,200, 1,201-1,300, 1,301-1,400, 1,401-1,500, 1,501-1,600, 1,601-1,700, 1,701-1,800, 1,801-1,900, 1,901-2,000, 2,001-2,100, 2,101-2,200, 2,201-2,300, 2,301-2,400, 2,401-2,500, 2,501-2,600, 2,601-2,700, 2,701-2,800, 2,801-2,900, 2901-3000, 3,001-3,500, 3,501-4,000, 4,001-4,500, 4,501-5,000, 5,001-5,500, 5,501-6,000, 6,001-6,500, 6,501-7,000, 7,001-7,500, 7,501-8,000, 8,001-8,500, 8,501-9,000, 9,001-9,500 or 9,501-9,776 probe molecules.

11. The method of claim 1, wherein said at least two variable region segments are selected from the group consisting of a V-segment, a D-segment and a J-segment.

12. The method of claim 11, wherein said V-segment has a third complementarity determining region specific portion which has an amino acid sequence selected from the group consisting of SEQ ID NOs: 1-23, and whereas each probe molecule of said probe set is substantially complementary to at least a portion of said sense or antisense strand of said nucleic acid sequence region of said specific polynucleotide wherein said portion of said sense or antisense strand encodes said third complementarity determining region specific portion of said V-segment.

13. The method of claim 1, wherein each probe molecule of said probe set is a single stranded polynucleotide composed of a number of nucleotides selected from a range of 24-48 nucleotides.

14. The method of claim 13, wherein said single stranded polynucleotide is a single stranded DNA molecule.

15. The method of claim 13, wherein said single stranded polynucleotide includes at least one nucleic acid sequence selected from the group consisting of SEQ ID NOs: 24-60 and antisense sequences thereof.

16. The method of claim 1, wherein the antigen receptor chain is a T-cell receptor chain.

17. The method of claim 16, wherein said T-cell receptor chain is T-cell receptor beta.

18. The method of claim 1, wherein the antigen receptor chain is a human antigen receptor chain.

19. A probe array comprising a support including a plurality of addressable locations and a probe set including a plurality of probe molecules, wherein each probe molecule of said plurality of probe molecules is attached to a specific addressable location of said plurality of addressable locations of said support, and is substantially complementary to a sense or antisense strand of a nucleic acid sequence region of a specific polynucleotide encoding a variant of an antigen receptor chain, said nucleic acid sequence region distinctly encoding a specific combination of at least two variable region segments of said antigen receptor chain.

20. The probe array of claim 19 wherein each distinct subset of a group of distinct subsets of distinct probe molecules of said probe set is attached to said probe array at a specific addressable location of said plurality of addressable locations, and includes a number of distinct probe molecules selected from a range of 1-128 distinct probe molecules.

21. The probe array of claim 19, wherein said probe array includes said plurality of addressable locations at a surface density of at least 625 specific addressable locations per square centimeter of said support.

22. The probe array of claim 19, wherein said probe set includes a number of probe molecules selected from the group consisting of 2, 3, 4, 5, 6, 7, 8, 9, 10, 11, 12, 13, 14, 15, 16, 17, 18, 19, 20, 21, 22, 23, 24 or 25 probe molecules.

23. The probe array of claim 19, wherein said probe set includes a number of probe molecules selected from a range of 26-30, 31-35, 36-40, 41-45, 46-50, 51-55, 56-60, 61-65, 66-70, 71-75, 76-80, 81-85, 86-90, 91-95, 96-100, 101-150, 151-200, 201-250, 251-300, 301-350, 351-400, 401-450, 451-500, 501-550, 551-600, 601-650, 651-700, 701-750, 751-800, 801-850, 851-900, 901-950, 951-1,000, 1,001-1,100, 1,101-1,200, 1,201-1,300, 1,301-1,400, 1,401-1,500, 1,501-1,600, 1,601-1,700, 1,701-1,800, 1,801-1,900, 1,901-2,000, 2,001-2,100, 2,101-2,200, 2,201-2,300, 2,301-2,400, 2,401-2,500, 2,501-2,600, 2,601-2,700, 2,701-2,800, 2,801-2,900, 2901-3000, 3,001-3,500, 3,501-4,000, 4,001-4,500, 4,501-5,000, 5,001-5,500, 5,501-6,000, 6,001-6,500, 6,501-7,000, 7,001-7,500, 7,501-8,000, 8,001-8,500, 8,501-9,000, 9,001-9,500 or 9,501-9,776 probe molecules.

24. The probe array of claim 19, wherein said at least two variable region segments are selected from the group consisting of a V-segment, a D-segment and a J-segment.

25. The probe array of claim 24, wherein said V-segment has a third complementarity determining region specific portion which has an amino acid sequence selected from the group consisting of SEQ ID NOs: 1-23, and whereas each probe molecule of said probe set is substantially complementary to at least a portion of said sense or antisense strand of said nucleic acid sequence region of said specific polynucleotide, wherein said portion of said sense or antisense strand encodes said third complementarity determining region specific portion of said V-segment.

26. The probe array of claim 19, wherein each probe molecule of said probe set is a single stranded polynucleotide composed of a number of nucleotides selected from a range of 24-48 nucleotides.

27. The probe array of claim 26, wherein said single stranded polynucleotide is a single stranded DNA molecule.

28. The probe array of claim 26, wherein said single stranded polynucleotide includes at least one nucleic acid sequence selected from the group consisting of SEQ ID NOs: 24-60 and antisense sequences thereof.

29. The probe array of claim 19, wherein said antigen receptor chain is a T-cell receptor chain.

30. The probe array of claim 29, wherein said T-cell receptor chain is T-cell receptor beta.

31. The probe array of claim 19, wherein said antigen receptor chain is a human antigen receptor chain.

32. A probe set comprising a plurality of probe molecules, each probe molecule of said plurality of probe molecules being substantially complementary to a sense or antisense strand of a nucleic acid sequence region of a specific polynucleotide encoding a variant of an antigen receptor chain, said nucleic acid sequence region distinctly encoding a specific combination of at least two variable region segments of said antigen receptor chain.

33. The probe set of claim 32, wherein the probe set includes a number of probe molecules selected from the group consisting of 2, 3, 4, 5, 6, 7, 8, 9, 10, 11, 12, 13, 14, 15, 16, 17, 18, 19, 20, 21, 22, 23, 24 or 25 probe molecules.

34. The probe set of claim 32, wherein the probe set includes a number of probe molecules selected from a range of 26-30, 31-35, 36-40, 41-45, 46-50, 51-55, 56-60, 61-65, 66-70, 71-75, 76-80, 81-85, 86-90, 91-95, 96-100, 101-150, 151-200, 201-250, 251-300, 301-350, 351-400, 401-450, 451-500, 501-550, 551-600, 601-650, 651-700, 701-750, 751-800, 801-850, 851-900, 901-950, 951-1,000, 1,001-1,100, 1,101-1,200, 1,201-1,300, 1,301-1,400, 1,401-1,500, 1,501-1,600, 1,601-1,700, 1,701-1,800, 1,801-1,900, 1,901-2,000, 2,001-2,100, 2,101-2,200, 2,201-2,300, 2,301-2,400, 2,401-2,500, 2,501-2,600, 2,601-2,700, 2,701-2,800, 2,801-2,900, 2901-3000, 3,001-3,500, 3,501-4,000, 4,001-4,500, 4,501-5,000, 5,001-5,500, 5,501-6,000, 6,001-6,500, 6,501-7,000, 7,001-7,500, 7,501-8,000, 8,001-8,500, 8,501-9,000, 9,001-9,500 or 9,501-9,776 probe molecules.

35. The probe set of claim 32, wherein said at least two variable region segments are selected from the group consisting of a V-segment, a D-segment and a J-segment.

36. The probe set of claim 35, wherein said V-segment has a third complementarity determining region specific portion which has an amino acid sequence selected from the group consisting of SEQ ID NOs: 1-23, and whereas each probe molecule of said probe set is substantially complementary to at least a portion of said sense or antisense strand of said nucleic acid sequence region of said specific polynucleotide wherein said portion of said sense or antisense strand encodes said third complementarity determining region specific portion of said V-segment.

37. The probe set of claim 32, wherein each probe molecule of said probe set is a single stranded polynucleotide composed of a number of nucleotides selected from a range of 24-48 nucleotides.

38. The probe set of claim 37, wherein said single stranded polynucleotide is a single stranded DNA molecule.

39. The probe set of claim 37, wherein said single stranded polynucleotide includes at least one nucleic acid sequence selected from the group consisting of SEQ ID NOs: 24-60 and antisense sequences thereof.

40. The probe set of claim 32, wherein said antigen receptor chain is a T-cell receptor chain.

41. The probe set of claim 40, wherein said T-cell receptor chain is T-cell receptor beta.

42. The probe set of claim 32, wherein said antigen receptor chain is a human antigen receptor chain.
